(19) United States
(12) Patent Application Publication
(10) Pub. No.: US 2013/0029321 A1
(43) Pub. Date: Jan. 31, 2013

(54) SAMPLE PROCESSING DEVICE AND METHOD

(71) Applicants: Mikkél Fougt Hansen, Vaerlose (DK); Christian Danvad Damsgaard, Farum (DK); Niels Bent Larsen, Rodovre (DK); Anders Wolff, Frederiksberg C (DK)

(72) Inventors: Mikkél Fougt Hansen, Vaerlose (DK); Christian Danvad Damsgaard, Farum (DK); Niels Bent Larsen, Rodovre (DK); Anders Wolff, Frederiksberg C (DK)

(73) Assignee: DANMARKS TEKNISKE UNIVERSITET, Lyngby (DK)

(21) Appl. No.: 13/577,744

(22) PCT Filed: Feb. 15, 2011
(86) PCT No.: PCT/DK11/50043

§ 371 Date: Oct. 15, 2012

Related U.S. Application Data
(60) Provisional application No. 61/304,583, filed on Feb. 15, 2010.

(30) Foreign Application Priority Data
Jul. 20, 2010 (EP) 10170124.1

(51) Int. Cl. B01L 3/00; C12Q 1/02; G01N 33/566; C12Q 1/70; C12Q 1/68; G01N 1/28

(52) U.S. Cl. 435/5; 422/500; 435/6.12; 436/175; 435/7.92; 435/29

(57) ABSTRACT

A sample processing device is disclosed, which sample processing device comprises a first substrate and a second substrate, where the first substrate has a first surface comprising two area types, a first area type with a first contact angle with water and a second area type with a second contact angle with water, the first contact angle being smaller than the second contact angle. The first substrate defines an inlet system and a preparation system in areas of the first type which two areas are separated by a barrier system in an area of the second type. The inlet system is adapted to receive a sample liquid comprising the sample and the first preparation system is adapted to receive a receiving liquid. In a particular embodiment, a magnetic sample transport component, such as a permanent magnet or an electromagnet, is arranged to move magnetic beads in between the first and second substrates.

### FIELD OF THE INVENTION

The present invention relates to a device and method for sample processing, and in particular the present invention relates to a device and method for sample processing using a patterned substrate.

### BACKGROUND OF THE INVENTION

Nucleic acid capture and extraction using magnetic beads is well established in laboratory and industrial settings and is also suited for use at the point-of-care (POC) in resource-limited settings. However, the devices presently proposed for use at the point of care are complex and expensive and mostly requiring specialized equipment and off-line sample preparation. Particularly, there is a need for the development of a simple procedure suitable for integration into a POC sample processing device for nucleic acid extraction from whole-blood in resource-limited settings. Moreover, immunoassays (e.g. for the detection of HIV) like the much used ELISA (Enzyme-Linked ImmunoSorbent Assay) require multistep sample processing, which can be a challenge in resource-limited settings. ELISA is used to measure antigen-antibody binding, and depending on the variation used, it will detect antigen (hormones, enzymes, microbial antigens, illicit drugs) or antibody (anti-HIV in the screening test for HIV infection) in body fluids.

It has previously been demonstrated that magnetic beads can be used to transport DNA from raw samples (whole blood, plasma, urine, throat swaps) through an oil phase with minimal carry-over of contaminants and that the purity of the DNA separated by this one-step procedure was sufficient to successfully carry out real-time PCR (Polymerase Chain Reaction), which is very sensitive to contaminants.

US 2009/0246782 A1 discloses devices and methods for performing biological reactions, and relates to the use of lipophilic, water immiscible, or hydrophobic barriers in sample separation, purification, modification, and analysis processes. The approach described in this reference, however, is relatively complex and not easily expanded to multiplexed operation and does not easily interface with multistep downstream detection assays.

Hence, an improved sample processing device would be advantageous, and in particular a more simple, efficient, cheap and/or reliable sample processing device which could relatively easily be expanded to multiplexed operation and interface with multistep downstream detection assays would be advantageous.

### SUMMARY OF THE INVENTION

It may be seen as an object of the present invention to provide a sample processing device that solves the above mentioned problems of the prior art.

It is a further object of the present invention to provide an alternative to the prior art.

Thus, the above described object and several other objects are intended to be obtained in a first aspect of the invention by providing a sample processing device comprising:
a first substrate, the first substrate having a first surface comprising at least two area types, a first area type with a first contact angle with water and a second area type with a second contact angle with water, the first contact angle being smaller than the second contact angle, and;
a second substrate, the second substrate having a second surface positioned substantially parallel with the first surface at a distance from the first surface of the first substrate;
   the first surface of the first substrate, or the first surface of the first substrate and the second surface of the second substrate, defines:
an inlet system provided in an area of the first type;
a first preparation system provided in an area of the first type; and
a barrier system provided in an area of the second type;
   wherein the inlet system and the first preparation system are separated by the barrier system, and wherein the inlet system is adapted to receive a sample liquid comprising the sample and the first preparation system is adapted to receive a receiving liquid.

The invention is particularly, but not exclusively, advantageous for obtaining an improved sample processing device, and in particular a more simple, efficient, cheap and/or reliable sample processing device which can relatively easily be expanded to multiplexed operation and be interfaced with multistep downstream detection assays.

Thus, the present invention provides a simplified sample processing device with reduced requirements for the surroundings, as compared to prior art sample processing devices.

In the present invention a simple, flexible and modular sample processing device for parallel sample pre-treatment and preparation is provided. The invention is suitable for both single-step extraction of a given sample, such as DNA, from complex samples, such as blood, and for multistep processing required for immunoassays. Moreover, the sample processing device is simple, inexpensive and effective.

The second surface is positioned substantially parallel with the first surface at a distance from the first surface of the first substrate, and it is understood that this distance may be a non-zero distance. In general this distance is understood to be a non-zero distance.

It is understood that both the first area type and the second area type of this embodiment may be hydrophilic. In a particular embodiment, the first and second area types of this embodiment are both hydrophilic. In another particular embodiment, the first and second area types of this embodiment are both hydrophobic. In another particular embodiment, the first and second area types of this embodiment are both hydrophobic and the second surface of the second substrate is hydrophilic so as to enable capillary forces to fill the appropriate systems of the sample processing device. In yet another particular embodiment, the surface chemistry of the first area type and the second area type are similar, but the topography of the first area type gives rise to a first contact angle with water and the topography of the second area type gives rise to a second contact angle with water. An advantage of this latter embodiment may be, that the step of chemically modifying the first and/or second area type may be omitted.

In embodiments, the present invention is embodied in the form of a plastic microfluidic chip comprising a first and second substrate, such as two flat plastic pieces, with a configurable structured modification of the surface chemistry to define hydrophilic and hydrophobic areas which can form fluidic pathways. In general, however, any material where the surface is suitable for structured modification of the surface chemistry and/or topography to define hydrophilic and hydrophobic areas can be applied, e.g. glass. The first and second area types define a wall-less two-phase fluidic design enabling aqueous phase systems, and possible also fluidic pathways, with sample-, washing- and reaction liquids, such as buffers, separated by a barrier system, such as air or an oil-phase. In the present context ‘chip’ is understood to be a small device arranged to enable a processing, such as a processing of sample in a sample liquid. Magnetic beads may be introduced to the sample liquid and used to capture and transport the sample, such as DNA or antigen, to the systems with the various washing buffers or reagents with minimal carry-over of contaminants. The sample processing device may utilize capillary forces for driving the movement of fluids in the microfluidics of the sample processing device, such as for filling, i.e. no external pumps are necessarily required; it is very flexible as the systems are defined by the structure of the surface modification, which can easily be changed; the operations are modular in the sense that extra operations such as washing or reaction steps can easily be added. In embodiments, the sample processing device is designed for multiplexed operation. The sample processing device can easily interface to subsequent detection steps. Thus, this approach is very suitable for the point-of-care (POC) use in resource-limited settings.

The sample processing device may be advantageous in that it might reduce a number of necessary washing steps in multistep sample processing by having minimal carry-over of liquid between the inlet system and the first preparation system.

It is generally understood that the first substrate and the second substrate are each of a solid material, such as solid material under ambient conditions, such as in atmospheric air and at standard ambient temperature (25 degrees Celcius) and pressure (103 kPa). It is understood that glass is a solid material. In an embodiment according to the invention, the sample processing device may be based on Cyclic Olefin Copolymer (COC) substrates. COC is oleophilic, hydrophobic and biocompatible. Hydrophilic structures on the COC substrates can be defined e.g. by oxygen (O₂) plasma treatment or stamping.

The approach of the present invention aims at achieving the efficient sample purification and concentration demonstrated by others using magnetic beads for DNA separation from an aqueous-phase sample through an oil-phase while also simplifying the practical operations and making the approach amenable to parallelization, multi-step processing and integration with other up- or down-stream sub-devices.

In another embodiment, the sample processing device may comprise or be attached to a characterization means so that the sample preparation, such as DNA preparation may be characterized. Such characterization may be quantitative PCR measurements, optical measurements (fluorescence and optical density measurements), and/or measurements using a radioactive label, such as a radioactive isotope. The samples may be solutions with known contents of PCR inhibitors spiked with DNA or more complex samples. This may be advantageous for testing purposes or for calibration purposes.

In another embodiment, there is presented a sample processing device wherein the first area type is hydrophilic and the second area type is hydrophobic. It is understood that embodiments where the first area type is hydrophilic and the second area type is hydrophobic are particular embodiments of the more general concept with a first substrate, the first substrate having a first surface comprising two area types, a first area type with a first contact angle with water and a second area type with a second contact angle with water, the first contact angle being smaller than the second contact angle. Where reference is made to first and second area type being hydrophilic respectively hydrophobic, it is thus understood that in a more general embodiment, the first and second area type may be a first area type with a first contact angle with water and a second area type with a second contact angle with water, the first contact angle being smaller than the second contact angle.

In another embodiment there is presented a sample processing device, wherein the difference in contact angle between the first contact angle of the first area type and the second contact angle of the second area type enables filling the inlet system and first preparation system with an aqueous solution and subsequent filling the barrier system with a liquid immiscible with the aqueous solution whereby the position of the aqueous phase and the liquid immiscible with the aqueous phase are defined by the positions of the first and second area types. An advantage of this may be that the positioning of the respective liquids in the respective systems may be done faster, more easily, more reliably and without taking regard to gravity.

In another embodiment, two systems in an area of the first area type, such as two preparation systems, may be fluidically connected via a channel area of the first area type, wherein the channel area is an area of the first type arranged so that an aqueous phase may be driven by capillary forces between the two systems while diffusion, such as diffusion of a sample and/or contaminant, between the two systems is negligible on a time scale comparable to a timescale of a processing on the sample processing device during use. In one particular embodiment, the channel area comprises a relatively thin area, such as the channel area being elongated in a direction between the two systems.

In a particular embodiment, the first surface of the first substrate and/or the second surface of the second substrate are substantially planar. In an embodiment the planar character of the first and/or second surface enables a particle to be transported in a plane parallel to the first and/or second surface from the inlet system, such as along a straight line or rectilinear path, through the barrier system and into the first preparation system. In a further embodiment, the first surface of the first substrate and/or the second surface of the second substrate are substantially parallel.

In some embodiments, the first surface of the first substrate has two area types and the second surface of the second substrate also has two area types, a first area type where the first surface is hydrophilic and a second area type where the first surface is hydrophobic. In a particular embodiment the pattern given by the locations of the first and second area type on the first surface on the first substrate is a mirror image of the pattern given by the locations of the first and second area type on the second surface on the second substrate. In another particular embodiment, the pattern given by the locations of the first and second area type on the first surface on the first substrate may be different from the mirror image of the pattern given by the locations of the first and second area type on the second surface on the second substrate, for example, an area of the first area type on the first surface of the first substrate may define the inlet system and an area of the first area type on the second surface of the second substrate may define the first preparation system.

In some embodiments, the first surface of the first substrate has two area types, a first area type where the first surface is hydrophilic and a second area type where the first surface is hydrophobic, and the second surface of the second substrate has only one area type, which may in a particular embodiment be the second area type, such as hydrophobic. An advantage of having the second surface of the second substrate being of the second area type may be that it limits spreading of the aqueous phase. In such embodiments the second surface of the second substrate generally has only one type of area, i.e., the surface properties of the second surface are substantially uniform. An advantage of having the second surface of the second substrate being substantially uniform may be that alignment with respect to the first surface of the first substrate is less critical. Another advantage may be that modification of only one surface is necessitated. Another possible advantage is that fabrication of the sample processing device may be kept simple.

In a particular embodiment, the first surface of the first substrate has two area types and the second surface of the second substrate also has two area types, a first area type where the first surface is hydrophilic and a second area type where the first surface is hydrophobic, so that the first surface of the first substrate defines an inlet system and the second surface of the second substrate defines a first preparation system.

In some further embodiments the first surface of the first substrate and/or the second surface of the second substrate is topographically structured without disabling the movement of magnetic beads within or between systems, such as blocking the fluidic pathway between the systems. An advantage of having a topographically structured first and/or second surface may be that it facilitates alignment of the first and second substrates. Another advantage may be that it substantially confines the spreading of the liquid in the second type of area to the lateral extents of the device. A further advantage may be that it enhances the stability of the separation of the aqueous phase and the gas phase, or the aqueous phase and the liquid phase immiscible with an aqueous phase by having structural protrusions or recesses on the first and/or second surface in accordance with the position of first area type and/or with the position of the second area type, or with the position of the boundaries of the first and second area types on the first surface of the first substrate.

Generally, the concept of a contact angle for a given surface is related to the angle that a liquid-vapour pair forms on the solid surface in question, i.e. the contact angle is a measurement performed in the common intersection line of the three phase system, the line is seen as a point in a conventional cross-sectional contact angle measurement setup. Typically, the liquid is chosen to be pure water (H₂0) and the measurement is performed at standard atmospheric temperature and pressure (SATP) for standardized measurements of hydrophilic or hydrophobic character of a surface. More generally, the contact angle is further related to the surface energy of the surface.

By hydrophilic surface is to be understood a surface which surface properties will, by definition, give rise to a contact angle of less than 90 degrees for a droplet of water applied to the surface. Often the concept of ‘wetting’ is also used to describe contact angles less than 90 degrees, typically close to 0 degrees.

By hydrophobic surface is to be understood a surface which surface properties will give rise to a water contact angle of 90 degrees or higher. It is generally understood, that most hydrophobic surfaces are also oleophilic, with a few exceptions, such as some fluorine-containing organic materials.

It should be noted that in general, the contact angle is influenced not only by the surface chemistry of the surface but also by the topology and/or morphology of the surface in question. Thus, for example the roughness of a surface may further influence the measured contact angle.

The sample liquid is understood to be an aqueous phase, which comprises the sample.

The receiving liquid is understood to be an aqueous phase. It is understood that water and water-based fluids are considered to be aqueous phases.

It is understood that in some embodiments, the inlet system and the first preparation system are separated by the barrier system, such as completely separated, or such as separated so that mixing of liquids or samples between the inlet system and the first preparation system may occur only on a timescale substantially larger than the timescale necessary for carrying out a sample processing.

Use of the word ‘system’ is in the following referred to and it is understood to cover any one of: inlet system, barrier system, first preparation system and any one of further preparation systems. It is understood that the size and location of each system is defined by the pattern of first- and second area types on the first substrate or the first substrate and the second substrate. In other words, each system substantially corresponds to a volume above the first surface, such as between the first surface and the second surface whose projection onto the first surface corresponds to an area of the first area type, or an area of the second area type.

In another embodiment according to the invention there may be provided a sample processing device, wherein the barrier system is adapted to receive a liquid immiscible with an aqueous phase. One advantage of having a barrier system which is adapted to receive a liquid immiscible with an aqueous phase may be, that it enables adding a liquid immiscible with an aqueous phase to the sample processing device, which liquid immiscible with an aqueous phase may substantially slow down evaporation of the sample liquid and/or the receiving liquid from the sample processing device. Another advantage of having a barrier system with a liquid immiscible with an aqueous phase, may be that the sample, during transport between the inlet system and the first preparation system may experience less degradation, such as less perturbation to its structure, such as less denaturation, with a proper choice of liquid filling the barrier system, compared to the barrier system being filled with a gas phase, such as atmospheric air. In particular, this may be the case if the sample comprises a molecule, such as a macromolecule, of biological origin. It is hypothesized that another advantage of having a barrier system with a liquid immiscible with an aqueous phase may be that the surface tension between the aqueous phase and the liquid immiscible with an aqueous phase may be less than a surface tension between the aqueous phase and a gaseous phase, such as atmospheric air. For a lower surface tension it may be possible to use less force when transporting a sample through the interface between the inlet system and the barrier system.

By ‘a liquid immiscible with an aqueous phase’, hereinafter interchangeably referred to as immiscible liquid, is to be understood a liquid which does not form a homogenous solution when mixed with an aqueous phase under ambient conditions, such as under conditions applicable for processing using the sample processing device. A property of immiscible liquids is that they cannot be diluted with water without separation, such as a property of immiscible liquids is that they cannot be diluted with at least an equal part of water without separation into more than one phase. In some embodiments, immiscible liquids have a low solubility for substances that interfere with a particular biological process such as nucleic amplification or biomolecule detection. In some embodiments, immiscible liquids have a low vapor pressure. Immiscible liquids tend to interact within themselves and with other substances through van der Waals forces. They have little to no capacity to form hydrogen bonds. Immiscible liquids typically have large o/w (oil/water) partition coefficients.

In another embodiment according to the invention, the liquid immiscible with an aqueous phase is chosen from the group of: oil, wax, ionic liquids, alcohols, amines, carboxylic acids, esters, amides, or ketones of a chemical structure containing a multitude of carbon atoms.

In a particular embodiment, there may be provided a sample processing device, wherein the liquid immiscible with an aqueous phase is chosen from the group of: oil, wax, ionic liquids. The liquid immiscible with an aqueous phase may also be chosen to be an organic compound comprising alcohols, amines, carboxylic acids, esters, amides, and/or ketones of a chemical structure containing a multitude of carbon atoms, such as a single compound or mixtures of compounds being of sufficiently low viscosity at the operating temperature to permit the transport of beads through their volume under the immiscible liquid. It is understood that oil, wax or ionic liquids may or may not comprise one or more of the functional groups mentioned above. The immiscible liquid component or components can, for example, be hydrocarbon-based liquids such as olefins, silicon-based oils such as poly(dimethyl siloxanes), halogenated oils such as perfluorocarbons or poly(chlorotrifluoroethylene), ionic liquids such as 1-butyl-3-methylimidazolium hexafluorophosphate, or any of a range of solvents exhibiting low miscibility with an aqueous phase, such as alcohols, amines, carboxylic acids, esters, amides, or ketones of a chemical structure containing a multitude of carbon atoms. Preferred immiscible liquids will have very low miscibility with an aqueous phase and will not adversely affect the bead-attached compounds being transported through the immiscible liquid.

In another embodiment according to the invention there may be provided a sample processing device comprising two or more preparation systems, each preparation system being separated by the barrier system. A possible advantage of having two or more preparation systems is that it enables a plurality of serial or parallel sample processing steps to occur. A sample may, for example, be moved from the inlet system to a first preparation system, and subsequently to a second preparation system. Each preparation system may be associated with a processing step, such as a purification step or a change to another liquid. In another exemplary embodiment, a plurality of samples may be moved from an inlet system to a plurality of different preparation systems, which preparation systems may be similar, which enables similar parallel processing. Alternatively, the preparation systems may not all be similar, which enables different processing steps to be carried out in parallel.

In another embodiment according to the invention there may be provided a sample processing device, wherein the first preparation system is pre-filled with a reagent. By pre-filling the first preparation system with a reagent, the sample processing device may be stored for later use, and upon filling with liquids, such as an aqueous phase, the reagents may be utilized without being added at the time of filling. Thus, adding of the reagents may be done in a period from fabrication of the sample processing device to use of the sample processing device. In another embodiment, a plurality of preparation systems are pre-filled with a reagent so as to enable more complex analysis to be carried out, without necessarily having to add a plurality of reagents when using the sample processing device.

In another embodiment according to the invention there may be provided a sample processing device, wherein the pre-filled reagent is chosen from the group comprising: a dried reagent, a freeze dried reagent, a reagent contained in a gel and a reagent contained in a liquid.

In another embodiment according to the invention there may be provided a sample processing device, wherein a sample may be moved from the inlet system to the first preparation system through non-solid matter, whereby the sample is moved along a trajectory substantially confined to a plane, such as confined to a plane, such as along a substantially straight line, such as the trajectory being confined within the inlet system, the barrier system and the first preparation system.

In another embodiment according to the invention there may be provided a sample processing device comprising a magnetic sample transport component arranged to move magnetic particles between the inlet system and the first preparation system and/or between two preparation systems.

This embodiment provides a modular wall-less fluidic sample processing device for sample preparation with magnetic sample control. This may be advantageous in that magnetic particles may be coated with a layer, such as a molecular layer, which may selectively bind certain samples of interest, such as certain molecules, nucleic acids or proteins. Thus by having a magnetic sample transport component, magnetic beads and hence a specific sample may be moved from the inlet system, through the barrier system and into the first preparation system.

It is within the capabilities of the skilled person to apply and control direction and magnitude of a magnetic field. In a particular embodiment, the magnetic sample transport component may be chosen from the group comprising: a permanent magnet and an electromagnet. The magnetic sample transport component may further be realized by structures of magnetic material adjacent to the first and/or second substrate, which structures of magnetic material are magnetized by an externally applied magnetic field.

The magnetic sample transport component may be provided in any suitable manner. It may in embodiments be motorized or operated by hand. In addition to capture and translation of beads, the magnetic manipulator may be provided to perform active manipulation of beads within a system to enhance the bead-sample interaction.

In another embodiment according to the invention there may be provided a sample processing device comprising a magnetic sample manipulation component arranged to move magnetic particles from a starting point to an end point along a path, the path being within the inlet system or within the barrier system or within the first preparation system, where the length of the path is substantially larger than a distance from the starting point to the end point. In particular embodiments the magnetic sample manipulation component is arranged to move magnetic particles in an oscillating motion. The magnetic sample manipulation component may include means applicable for controlling a magnetic field. A possible advantage of having a magnetic sample manipulation component is that is enables enhanced mixing of the beads with the liquid. An advantage of this may be that it enhances the interaction between the beads and the liquid in an area in the sample processing device, so that the beads may interact with a larger volume of liquid. Another possible advantage is that it effectively reduces the time needed for the magnetic beads to bind to the sample. An advantage of this may be that the time for performing a process, such as an analysis, may be reduced.

The magnetic manipulator may be provided in any suitable manner. It may in embodiments be motorized or operated by hand. In addition to mixing of the beads with the liquid in the sample processing device, the magnetic manipulator may be provided to enable moving of magnetic beads within a system or between systems.

In another embodiment according to the invention there may be provided a sample processing device comprising a fluid reservoir connected to any one of: the inlet system, the first preparation system, the barrier system, and wherein the inlet system, the preparation system and/or the barrier system is dimensioned so that fluid is pulled from the fluid reservoir to the inlet system, the preparation system and/or the barrier system by means of capillary forces. An advantage of this may be that if more liquid is applied than is needed to fill any one of the systems, then the excess liquid will remain in the reservoir. The system connected to the reservoir, may consequently effectively fill itself with the correct amount of liquid, and it is hence not necessary to meticulously measure the correct amount of liquid. Another advantage may be that it minimizes the risk of applying too little liquid to fill a given system properly. Another advantage may be that a pump may not be needed.

In another embodiment according to the invention there may be provided a sample processing device, wherein the barrier system is fluidically connected to a least one venting means, the venting means allowing passage of a fluid from within any one of:
the inlet system,
the barrier system,
the first preparation system,
   and through the first substrate and/or through the second substrate and/or between the first substrate and the second substrate.

The venting means may be through-going holes, referred to hereinafter as venting holes, which may be located towards the end of each of the intended fluidic pathways. The venting holes may have hydraulic diameters in the range of 1 micrometer to 10 millimeters, with further preferred hydraulic diameters in the range of 100 micrometers to 5 millimeters.

In a specific embodiment, the venting means are through going holes in the first substrate and/or the second substrate.

An advantage of having venting means may be that the sample processing device may be filled faster. Another possible advantage is that small air pockets may not form in the sample processing device.

According to a second aspect of the invention, the invention further relates to a method of processing a sample on a sample processing device, the sample processing device comprising:
a first substrate, the first substrate having a first surface comprising two area types, a first area type with a first contact angle with water and a second area type with a second contact angle with water, the first contact angle being smaller than the second contact angle;
   the first substrate defines:
an inlet system provided in an area of the first type;
a first preparation system provided in an area of the first type; and
a barrier system provided in an area of the second type;
   wherein the inlet system and the first preparation system are separated by the barrier system, and wherein the inlet system is adapted to receive a sample liquid comprising the sample and the first preparation system is adapted to receive a receiving liquid, the method comprising:
providing the sample liquid comprising a sample in the inlet system;
providing the receiving liquid in the first preparation system; and
moving the sample through the barrier system to the first preparation system to generate a processed sample.

This aspect of the invention is particularly, but not exclusively, advantageous in that the method according to the present invention may be implemented with relatively simple equipment. Other possible advantages may include that the method according to the invention enables simple, efficient, cheap and/or reliable sample processing which can relatively easily be expanded to multiplexed operation and be interfaced with multistep downstream detection.

The method according to the invention may thus provide a method for simple, flexible and modular sample processing, for parallel sample pre-treatment and preparation. The method according to the invention may furthermore be suitable both for single-step extraction of a given sample, such as DNA, from complex samples, such as blood, and for multistep processing required for immunoassays. Moreover, the method according to the invention may be simple, inexpensive and effective.

It is contemplated that embodiments where the second surface of the second substrate defines the inlet system or the first preparation system is also to be comprised within the scope of the invention.

In a further embodiment according to the invention there is provided a method of processing a sample on a sample processing device, wherein the first surface is hydrophilic in the first area type and the first surface is hydrophobic in the second area type.

In another embodiment according to the invention there may be provided a method of processing a sample on a sample processing device, wherein the inlet system comprises magnetic beads with associated molecules, and wherein the movement of the sample through the barrier system is done by moving the magnetic sample transport component to move the sample through the barrier system. An advantage of this may be that sample control can be obtained with simple and reliable means.

In another embodiment according to the invention there may be provided a method of processing a sample on a sample processing device, wherein prior to moving the sample through the barrier system, a liquid immiscible with an aqueous phase is provided in the barrier system. An advantage of this may be that the evaporation of liquids from the sample processing device is slowed down. It is understood that in particular embodiments, the liquid immiscible with an aqueous phase is provided in the barrier system, so as to span an area between the inlet system and the first preparation system. An advantage of this may be that a sample may be transported from the sample liquid in the inlet system, through the liquid immiscible with an aqueous phase in the barrier system and into the receiving liquid in the first preparation system and throughout this transport being immersed in liquid and not exposed to air.

In another embodiment according to the invention there may be provided a method of processing a sample on a sample processing device, the method further comprising moving the processed sample through the barrier system from the first preparation system to a second preparation system to generate a further processed sample.

In another embodiment according to the invention there may be provided a method, wherein moving the sample is done along a trajectory, the trajectory being substantially confined to a plane, such as confined to a plane, such as along a substantially straight line such as, such as along a rectilinear path.

In another embodiment according to the invention there may be provided a method of processing a sample on a sample processing device, wherein the sample contains at least one component from the group of: cells, intact cells, vira, nucleic acids, peptides, proteins, small organic molecules, and small organic molecules being toxic to any one of environment, animals, plants, and/or humans.

Small organic molecules may for example comprise environmental poison.

It is understood, that the sample may be a biological sample.

In an embodiment according to the invention there may be provided a method of processing a sample on a sample processing device, wherein the sample is a biological sample.

The first aspect and the second aspect of the present invention may each be combined with the other aspect. These and other aspects of the invention will be apparent from and elucidated with reference to the embodiments described hereinafter.

### BRIEF DESCRIPTION OF THE FIGURES

The sample processing device and method according to the invention will now be described in more detail with regard to the accompanying figures. The figures show one way of implementing the present invention and is not to be construed as being limiting to other possible embodiments falling within the scope of the attached claim set.

FIG. 1 illustrates a demonstrator sample processing device for two-phase fluid control,

FIG. 2 illustrates an embodiment procedure for sample handling and extraction,

FIG. 3 schematically illustrates another embodiment procedure for sample handling and extraction,

FIG. 4 shows other demonstrator sample processing devices,

FIG. 5 shows an exemplary movement of magnetic particles,

FIG. 6 shows a schematic of magnetic bead translation,

FIG. 7 shows a side view schematic of magnetic bead capture,

FIG. 8 shows a schematic with various lengths indicated,

FIG. 9 shows a schematic illustration of a standard ELISA procedure according to the prior art,

FIG. 10 shows a schematic illustration of a sample processing device based implementation of a standard ELISA procedure for single sample analysis,

FIG. 11 shows a schematic illustration of a sample processing device based implementation of a standard ELISA procedure for multiple sample liquids, single sample analysis,

FIG. 12 shows a schematic illustration of a sample processing device based implementation of a standard ELISA procedure for multiple sample liquids, multiple sample analysis,

FIG. 13 shows a perspective schematic drawing of an embodiment of a sample processing device according to an embodiment of the invention.

### DETAILED DESCRIPTION OF AN EMBODIMENT

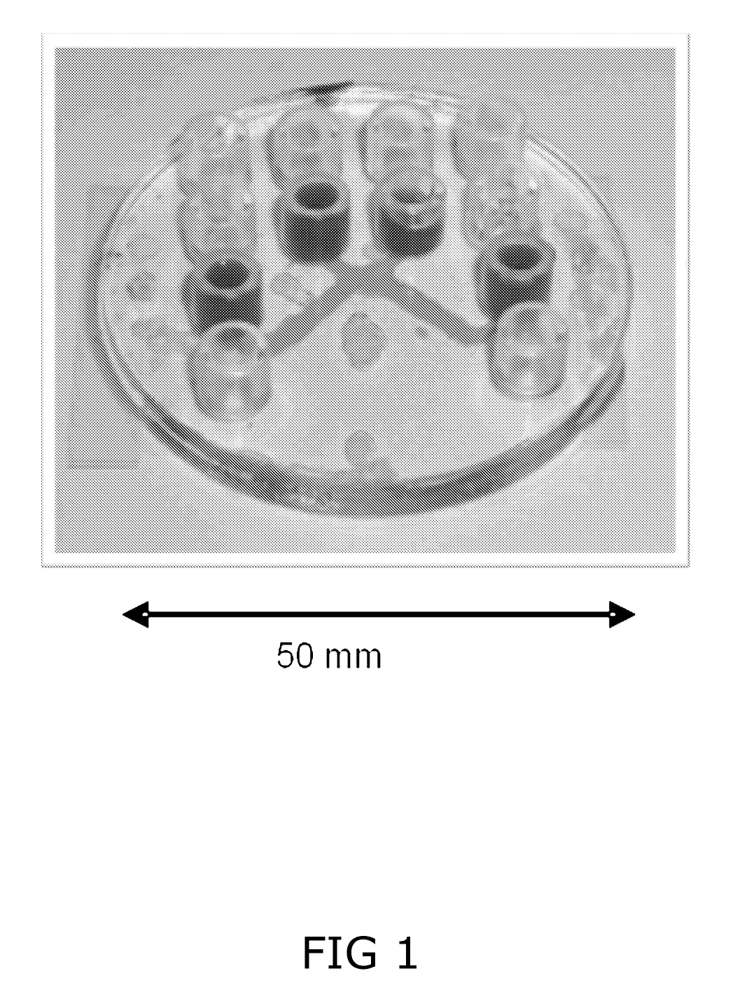

FIG. 1 illustrates a demonstrator sample processing device for two-phase fluid control. Hydrophobic plastic substrates made by injection molding of cyclic olefin copolymer (COC) have been made hydrophilic in a cross-shaped geometry by exposure of the opposing COC surfaces to an air plasma through mask openings having the intended cross-shaped geometry; dyed water (grey) has been introduced through well openings located above the hydrophilic areas followed by introduction of clear oil in the other wells. The plastic substrates are separated by use of two pieces of polymer sheet acting as spacers, placed between the plates in the rim region.

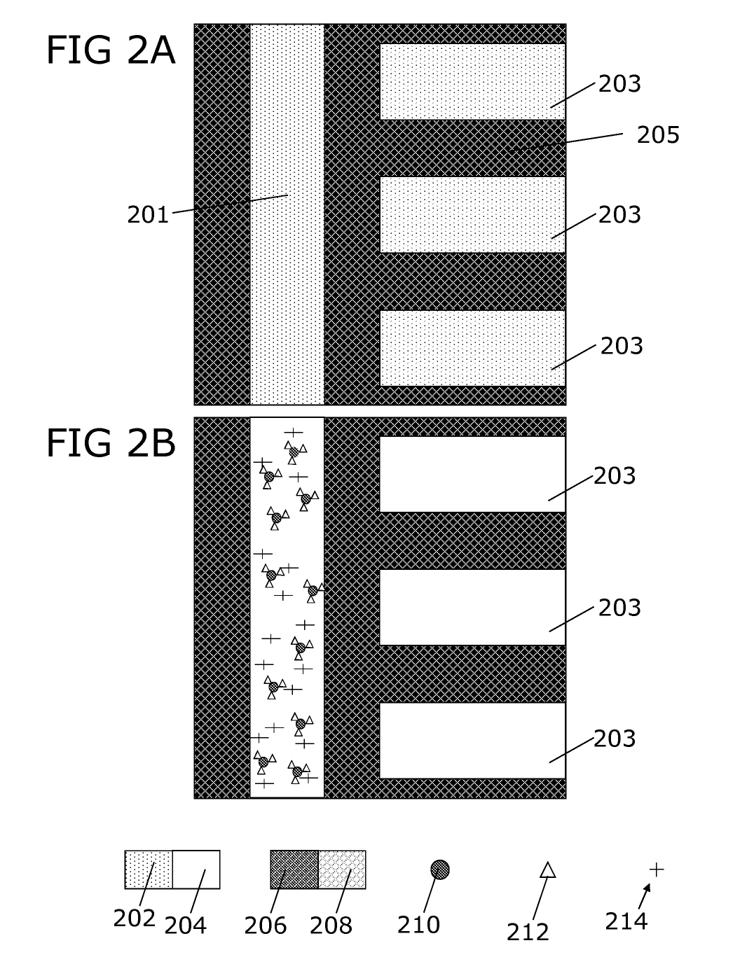

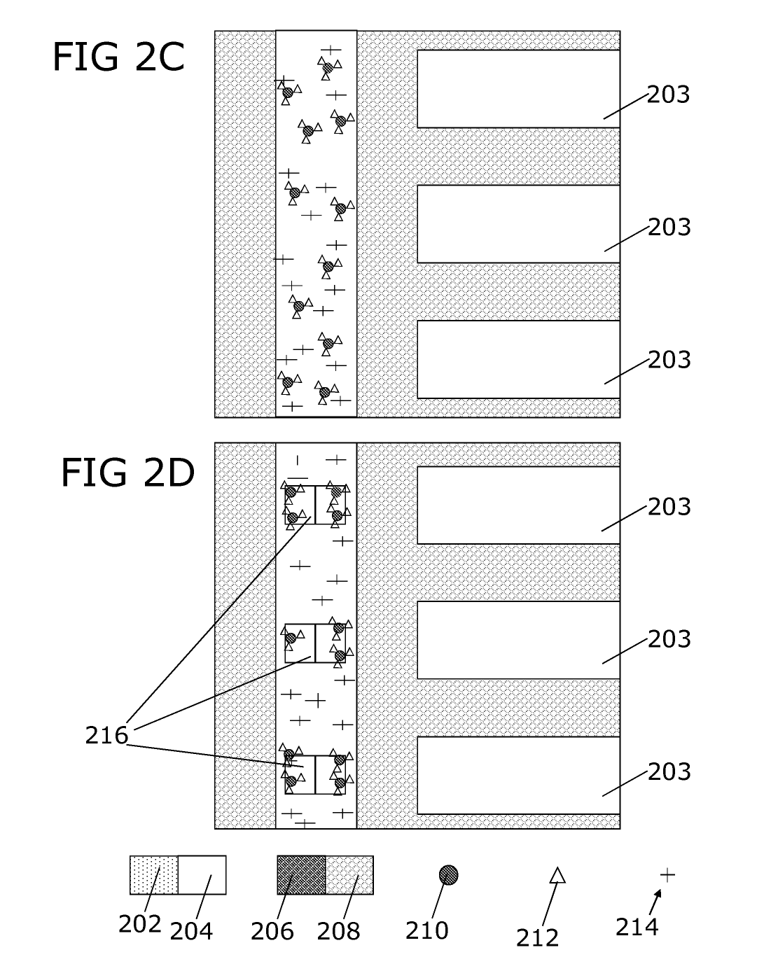

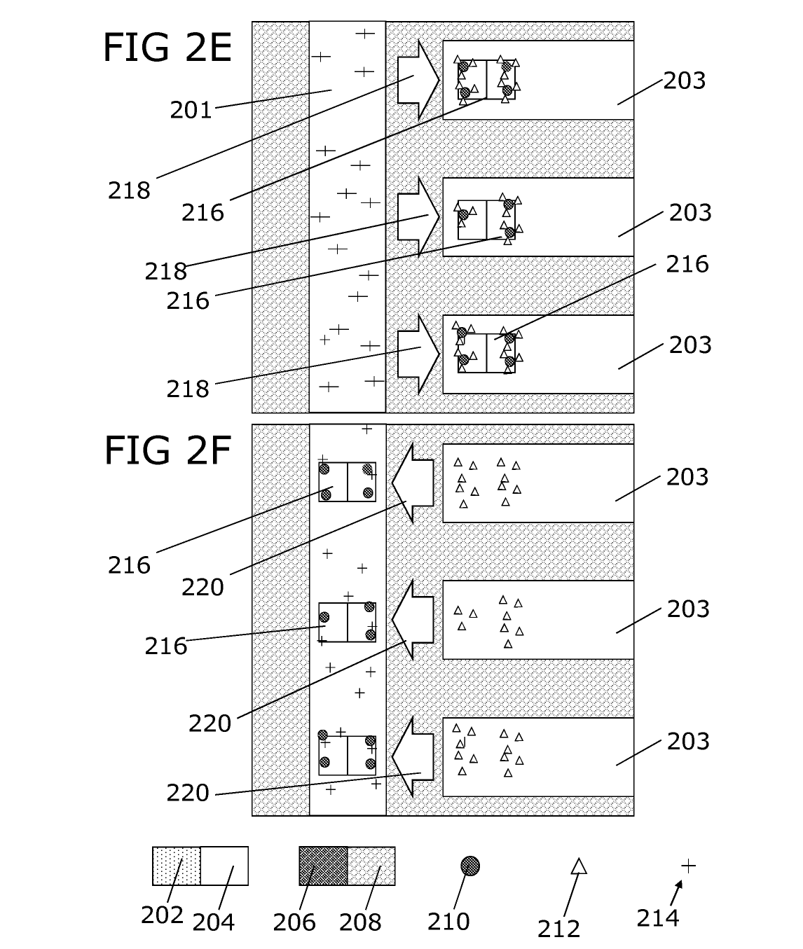

FIG. 2 schematically illustrates an embodiment procedure for sample handling and extraction by use of magnetic bead transportation in a wall-less two-phase fluidic sample processing device. The width of each system is on the order of 1 mm. The sample processing device is suitable for parallel preparation.

FIG. 2A shows the top view of a first surface of a first substrate of a sample processing device consisting of flat substrates with a structured surface modification such that hydrophilic and hydrophobic systems are defined and connected to fluid reservoirs (the fluid reservoirs are not shown). In FIG. 2A there are no liquids present, i.e., the sample processing device is in dry condition. Notice that the different schematic elements of FIG. 2 are illustrated below FIG. 2B, below FIG. 2D, and below FIG. 2F. The figure shows a hydrophilic area, hereinafter also referred to as an S1 type area, in dry condition 202, an S1 type area, i.e., a hydrophilic area, in wet condition 204, a hydrophobic area, hereinafter also referred to as an S2 type area, in dry condition 206, an S2 type area, i.e., a hydrophobic area, in wet condition 208. Furthermore is shown a magnetic bead 210, a specific sample 212 and a non-specific sample 214. The same surface modifications are also found in the liquid filled sample processing device of FIGS. 2B-2F even though the surface structures are not directly illustrated. FIG. 2A also shows a pattern on the structured first surface whereby a number of systems are defined, such as a first preparation together with a plurality of preparation systems 203. The figure also shows an inlet system 201 in a dry S1 type area and a barrier system 205 in a dry S2 type area.

FIG. 2B shows that aqueous phases (release buffers and sample liquid mixed with magnetic beads) are introduced in the reservoirs and drawn into the system by capillary forces. The hydrophobic surface will prevent the aqueous phases from entering the S2 type areas.

FIG. 2C shows that oil is introduced into one or more reservoirs as needed and drawn into the sample processing device so as to fill the barrer system by capillary forces.

FIG. 2D shows that magnets 216 are provided to capture the magnetic beads with attached molecules, e.g. attached DNA molecules. The magnets are placed on the other side of the first and/or second substrate so as to avoid physical contact with either of the liquid phases.

FIG. 2E shows that the magnetic beads are then translated by magnetic forces in the direction indicated by arrows 218 through the oil phase into the release buffers in preparation systems 203 (containing e.g. different primers for PCR amplification) leaving impurities dissolved in the water behind.

FIG. 2F shows that the captured molecules are released, e.g. by an increase in the temperature, and the magnetic beads can optionally be removed, such as by translating the magnetic beads by magnetic forces in the direction indicated by arrows 220 through the oil phase back into the inlet system. The resulting purified sample can then be further processed or detected in a downstream detection assay.

The sample processing device may comprise a first and a second substrate with opposing surfaces separated by a distance h. The surface of one or both of the substrates may have been modified (e.g. physically or chemically) in a pattern such that two types of surfaces are available, the two types having different properties:
S1: hydrophilic (wettable by aqueous phases)
S2: hydrophobic (in one embodiment of the invention the S2 surface is wettable by a second liquid, the second liquid being immiscible or having low miscibility with aqueous phases, hereinafter referred to as immiscible liquid (IL). In another embodiment of the invention the S2 surface holds no liquid and is surrounded by a gas phase, such as the ambient atmosphere)

The second liquid, IL, which has low miscibility with aqueous phases, can be a single compound or mixtures of compounds being of sufficiently low viscosity at the operating temperature to permit the transport of beads through their volume under the IL. The IL component or components can, for example, be hydrocarbon-based liquids such as olefins, silicon-based oils such as poly(dimethyl siloxanes), halogenated oils such as perfluorocarbons or poly(chlorotrifluoroethylene), ionic liquids such as 1-butyl-3-methylimidazolium hexafluorophosphate, or any one of a range of solvents exhibiting low miscibility with aqueous phases, such as alcohols, amines, carboxylic acids, esters, amides, or ketones of a chemical structure containing a multitude of carbon atoms. Preferred IL phases will have very low miscibility with aqueous phases and will not adversely affect the bead-attached compounds being transported through the IL phase.

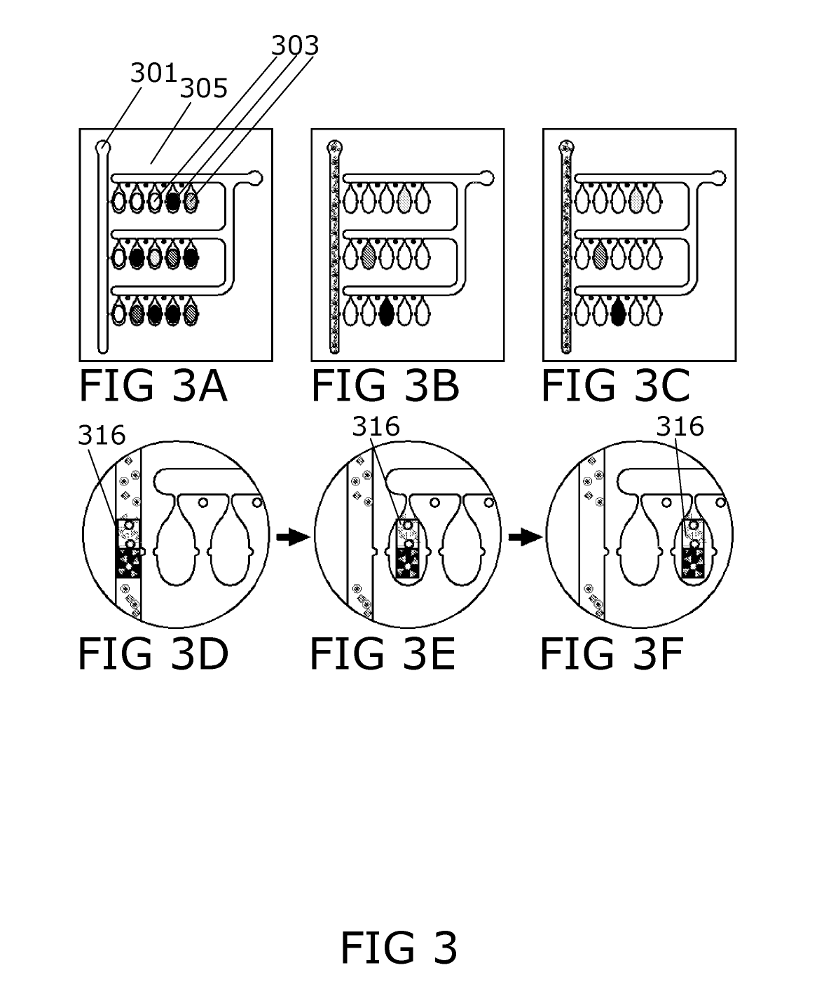

The pattern of the surface modification is chosen such that an S1 pathway, such as hydrophilic channel areas of the first area type (designated sample channel) is connected to a sample liquid inlet and a network of one or several hydrophilic pathways, such as hydrophilic channel areas, are connected to one or several inlets for aqueous phases and/or aqueous phase-based reagents (designated reagent channels) as schematically illustrated in FIG. 2A. The sample channel and the reagent channels are separated by a barrier system, which barrier system is realized by the S2 areas. The reagent channels may contain reagents in dry form such that these reagents are dissolved when an aqueous phase is introduced. A design showing sample channels and reagent channels is also shown in FIG. 3.

Under and/or over the first substrate and/or the second substrate, there is a magnetic sample transport component and/or a magnetic manipulation component. In one embodiment, the magnetic sample transport component and/or a magnetic manipulation component may comprise a permanent magnet, an electromagnet or magnetic structures of other means capable of producing a magnetic field. An advantage of having an electromagnet may be that an electromagnet may be switched on and off. The permanent magnet or electromagnet can be moved so as to move magnetic beads between the first substrate and the second substrate. The magnetic beads may be moved by means of the magnetic sample transport component, such as moved from one area to another, such as from the inlet system to the first preparation system through a barrier system. Alternatively, the magnetic beads may by moved within an area, such as within the inlet system, the barrier system or the first preparation system, so as to induce stirring, such as magnetically induced stirring.

In one embodiment the magnetic sample transport component and/or the magnetic manipulation component may comprise a varying magnetic field, controlled so as to enable magnetic control over magnetic beads without having moving parts, such as moving magnets. In one embodiment, the magnetic sample transport component and/or the magnetic manipulation component comprises a plurality of electromagnets or magnetic structures.

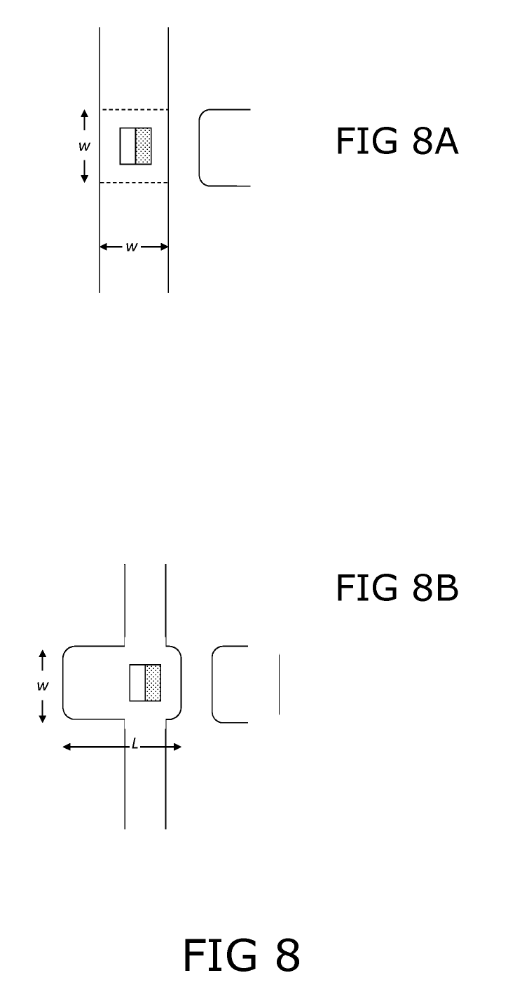

The separation between the two substrates can be in the range from h˜10 micrometer to h˜5 mm. It is dictated by the ability to fill the inlet system and the preparation system by capillary forces, to control the two liquid phases (such that gravity does not significantly shift the fluid boundaries) and to control the magnetic beads. The main limiting factor of these is likely the magnitude of capillary forces. A typical separation between the two substrates is h=1 mm, and typical widths of the S1 pathways and S2 areas separating S1 pathways are in the range w=2-5 mm (see FIG. 8 for a schematic illustration of the lengths). The total sample processing device size can be chosen to be similar to a microscope slide (such as 26×76 mm²). The thickness of each of the first and/or second substrate is typically 5 mm or less, but is only limited by the requirement for manipulating beads by external fields. A typical thickness will be from 0.5-2 mm for at least one of the substrates. Large thicknesses impose limitations on the magnitude and spatial localization of the magnetic forces, while small thicknesses may affect the mechanical stability of the final sample processing device. It is realistic to achieve significant magnetic forces on beads in the systems through a 0.25 mm thick substrate by use of a cubic permanent magnet with side lengths of about 2-4 mm. The first and second substrate may be of different thickness. Thus, a preferred embodiment will employ one substrate of millimeter-range thickness, typically 0.5 mm-2 mm, to ensure mechanical stability while the other substrate will be of substantially smaller thickness, typically 10 micrometer-0.5 mm, to facilitate accurate manipulation of beads by externally applied magnetic fields.

The first and second substrate can be made of the same material or of two different materials. Advantageously, the material to be used for the first and/or second substrate has properties so that
one surface must allow for surface patterning, so as to enable a pattern with both S1 and S2 surface areas,
at least one substrate material must allow for external manipulation of beads in the assembled two-substrate construct,
at least one substrate material must allow for read-out of an analytical signal for two-substrate constructs with an integrated analysis process.

At least one substrate material will preferably be non-magnetic to facilitate manipulation of beads by external fields. In a preferred embodiment, both substrate materials will be non-magnetic. In an embodiment at least one of the first substrate and the second substrate will have such optical transmittance that optical analysis through at least one of the first substrate and the second substrate will be possible. In a further preferred embodiment, both materials will have such optical transmittance that optical analysis through the substrates will be possible. Preferred materials include organic polymer materials and inorganic dielectrics of substantially high optical transmittance. Preferred organic polymer materials include polyolefins such as polystyrene, polypropylene, polyethylene, cyclic olefin copolymer, poly(butadiene), poly(isoprene), and copolymers of these, polymethacrylates such as poly(dimethyl methacrylate), polycarbonates such as bisphenol A polycarbonate, polyesters such as poly(ethylene terephtalate), polyurethanes such as thermoplastic polyurethanes, and silicones such as poly(dimethyl siloxane). Preferred inorganic dielectrics include silicon-based glass.

The first and second substrate can be fabricated by standard processing technologies for shaping of polymer or inorganic materials, e.g. glass or ceramic materials, known to those skilled in the art. Processing technologies for shaping polymer substrates include injection moulding of polymer granulate and cutting, milling, or ablation of extruded, calendered or moulded polymer plates. Surface structuring of cut, milled, or ablated items may occur during the former processing, or in subsequent processing steps, e.g. by hot embossing. Processing technologies for shaping glass or ceramic materials include cutting, milling, or ablation of extruded, calendered, or moulded plates. Surface structuring of cut, milled, or ablated items may occur during the shaping process or in subsequent processing steps.

Advantageously, first and second substrate are given so that
their surface structure will not obstruct the motion of the beads when manipulated by magnetic fields, and
their inner surface can be patterned with surface properties S1 and S2.

Surface properties of surface areas S1 and S2 may result from chemical surface properties, or from a combination of chemical and topographical surface properties. Hydrophilic surface properties will, by definition, give rise to a contact angle of less than 90 degrees for a droplet of water applied to the surface. Hydrophobic surface properties will give rise to a water contact angle of 90 degrees of higher. Surface areas S1 or S2 may be inherent to the substrate material. For substrate materials chosen from the classes of polyolefins, fluorinated polymers or silicones, the clean material surface will exhibit properties corresponding to S2 areas. For substrate materials chosen from the classes of polymethacrylates, polycarbonates, polyesters, polyurethanes, or glass, the clean material surface will typically exhibit properties corresponding to S1 areas.

S1 area properties may be induced on a nominally S2 area by exposure to oxidizing environments, such as oxidizing liquids, e.g. concentrated nitric acid, ozone, plasma, corona, or flame treatment. S1 area properties may further be induced by deposition of chemistry with S1 area characteristics such as plasma-assisted deposition of polymers, e.g. poly(vinyl pyrrolidone) or oligo(glymes), by photochemical coupling, such as UV-light induced coupling of oligo(ethylene oxide)-modified anthraquinones, by physical vapour deposition, chemical vapour deposition, or printing of materials, e.g. via screen or tampon printing, with S1 area characteristics, or by wet chemical processes that couple chemistry with S1 area characteristics to the surface.

S2 area properties may be induced on a nominally S1 area surface by deposition of chemistry with S2 area characteristics such as plasma-assisted deposition of polymers, e.g. poly(trifluoro methylene), poly(octafluorocyclobutane), or polystyrene, by photochemical coupling, e.g. of benzophenone, by physical vapour deposition, chemical vapour deposition, or printing of materials, e.g. via screen or tampon printing, with S2 area characteristics, or by wet chemical processes that couples chemistry with S2 area characteristics to the surface, e.g. reaction of trichloroperfluorodecylsilane with glass.

Patterning with S1 area or S2 area type properties may occur by exposure to the reagents through a physical mask protecting other surface areas, by patterned light exposure for processes involving photochemical processes, or may be inherent to the modification process as for screen printing and tampon printing.

Topographical surfaces may be introduced in S1 areas, in S2 areas, or in both types of areas to make the respective areas more hydrophilic or more hydrophobic by means of increasing the ratio of the effective (developed) surface area to the projected surface area above unity. Surface structures may also be introduced to act as partial physical barriers between liquid phases, such as to reduce the interfacial area between neighbouring phases while retaining a path for beads to be transported between and across neighbouring phases. In a particular embodiment, the first substrate and/or the second substrate may be designed and fabricated so as to have partially walled-off the interface between neighbouring phases, such as between an S1 area and an S2 area, thus stabilizing the interfacial separation, while still having a through going hole in the wall, such as at the bottom of the wall, such as a through going hole in the wall, which through going hole lies in the plane of the first surface of the first substrate and/or the second surface of the second substrate, which through going hole is dimensioned so as to allow beads to pass through the through going hole.

The two substrates may be separated by a spacer that defines the height of the systems and bonded (e.g. by laser welding or ultrasonic welding). For the fluid control, it may not necessarily be critical that the sample processing device is leak-tight.

In the following, an exemplary method of processing a sample, on a sample processing device as described above, is described with reference to FIG. 2. The method may comprise the following steps, however, it may also comprise additional steps, and it is also understood that steps which are described may be left out.

FIG. 2A describes preparation for use of the sample processing device. Prior to use, the systems of the sample processing device can be loaded with dry reagents. This will make it possible to have several reagents that can be prepared with just one infusion of an aqueous phase. Alternatively, several reagents can be introduced through separate inlets. The sample processing device is assumed to be in an initial dry state

FIG. 2B describes capillary filling of preparation systems, such as the first preparation system, and inlet system. First, an aqueous phase, which in the present example is water, is introduced to the preparation systems. In practice, water or the aqueous-based reagents can be introduced into one or more wells on top of the sample processing device, such as through going holes in the first surface and/or the second substrate and/or through the side of the sample processing devices, such as between the first and second substrate. The water will then be sucked into the preparation systems by capillary forces and it will stop when it reaches S2 areas. The sample processing device may need pressure relief holes, such as venting means, such as venting holes (not indicated in FIG. 2), such that no pressure is built up that could prevent more fluid from entering the sample processing device.

Then (or simultaneously), the sample is introduced to the inlet system, e.g. through a well on top of the sample processing device, and the inlet system is filled by capillary forces. The sample can be premixed with magnetic beads or dried magnetic beads can be placed in the inlet system or in reservoirs near the inlet system. Depending on the desired functionality of the sample processing device, one or more different magnetic bead types can be used, such as one or more magnetic beads coated with different material, such as different molecular layers, so as to be capable of selectively binding different specific samples.

FIG. 2C describes capillary filling of a liquid immiscible with an aqueous phase. It is noted, that this step may in some embodiments be left out as the barrier system, which in this embodiment is filled with a liquid immiscible with an aqueous phase, may in some embodiments instead be filled with a gas, such as the ambient, atmospheric air. It is noted, that if a gas is used, this will not be driven by capillary forces, but rather by diffusion and pressure. If needed, the liquid immiscible with an aqueous phase (e.g. oil), which is immiscible with water, is introduced through an inlet. This phase will wet and fill the S2 areas. If needed, pressure relief holes can be introduced to avoid areas that are not filled due to pressure build-up. Note, that advantageously, this phase is filled after the steps depicted in FIGS. 2A-2B as it may otherwise wet the hydrophilic S1 areas as well.

FIG. 2D describes magnetic bead capture. The magnetic beads with attached sample are now captured using a magnetic field. If a permanent magnet is used it can be placed either under or over the sample processing device and the strength of the magnetic capturing force can be varied, such as diminished by moving it away from the sample processing device, such as enhanced by bringing it closer to the sample processing device.

The use of two magnetic structures (one below and one above the sample processing device) will make it possible to move the beads up and down inside the systems to enhance the mixing between the beads and the fluid in a system. This can enhance the speed of the magnetic capture of the sample, but it can also be beneficial for enhancing the efficiency of e.g. washing or detection steps (it will correspond to a stirring of the magnetic beads). This approach to mixing is known in the literature and has been described. Alternatively, a single magnet moved from one side of the sample processing devices to the other may also enable moving of the beads up and down inside the systems to enhance the mixing between the beads and the fluid in a system.

FIG. 2E describes magnetic bead translation. A translation of the magnetic structures will move the beads from the inlet system 201 to the preparation systems 203 in FIG. 2E. The liquid immiscible with an aqueous phase will work as an immiscible phase filter and ensure that there is a minimal carry-over of non-specific samples and contaminants from the inlet system 201.

Several serial processes can be carried out by repeating the steps depicted in FIGS. 2D-2E.

FIG. 2F describes sample release and magnetic bead removal. It is noted that this step is optional. This step is included to illustrate the release of the captured sample (if needed). In the case of DNA the sample release/elution could be carried out thermally by increasing the temperature above the melting point of double-stranded DNA or it could be induced by a change of the chemical conditions (e.g. salt concentration). Subsequently, the beads can be removed, if needed (for example if their presence inhibits the reactions to take place).

The illustration in FIG. 2 would be suitable for sample preparation for on-chip PCR (polymerase chain reaction), such as PCR on an embodiment of the sample processing device according to the invention. In this case, the solutions in preparation systems 203 could be PCR buffer solutions containing different primers for real-time PCR analysis of different samples.

FIG. 3 schematically illustrates another embodiment procedure for sample handling and extraction by use of magnetic bead transportation in a wall-less two-phase fluidic sample processing device. The sample processing device combines parallel serial preparation aspects to provide a sample processing device for multiplexed, multistep operations.

FIG. 3A shows a flat first substrate, embodied in the present example by a plate with a structured chemical surface modification by defining hydrophilic areas on an otherwise hydrophobic substrate. The hydrophilic areas and the hydrophobic area are connected to fluid reservoirs (not shown). The sample processing device comprises a number of preparation systems 303 comprising various reagents, as indicated by the different spots. In an embodiment, these are freeze-dried on-substrate. However, reagents may be provided in any suitable way. The figure shows an inlet system 301, a barrier system 305 in an S2 type surface area, a plurality of preparation systems 303 which in the present figure corresponds to the number of reservoirs comprising various reagents, as indicated by the different spots. In FIG. 3A there are no liquids but there are freeze-dried reagents.

In FIG. 3B shows capillary filling of a sample liquid, which sample liquid comprises the sample and beads, the sample liquid is filled into the inlet system 301. An aqueous phase, which aqueous phase in the present example is water, is introduced through one inlet into the first preparation system and further preparation systems 303 and the reagents dissolve in their respective preparation systems 303, and the sample, premixed with magnetic beads, is introduced through another inlet, such as the inlet to the inlet system. Both liquids are drawn into the system by capillary forces. It is understood that a change in color and/or transparency of the respective preparation systems 303 may change upon introduction of water, such as dependent on the optical properties of the pre-filled reagents.

In FIG. 3C, the oil-phase is introduced and drawn into the olephilic area of the barrier system 305 of the system by capillary forces while air escapes through venting holes and/or through the sides of the sample processing device.

FIG. 3D shows magnetic bead capture by means of a magnet 316.

FIG. 3E shows first magnetic bead translation by means of a magnet 316.

FIG. 3F shows second magnetic bead translation by means of a magnet 316.

FIGS. 3D-3F illustrate the principle of the sample capture by magnetic beads and translation of the magnetic beads with the captured sample through the oil-phase between the various washing and reagent solutions in the preparation systems. For PCR nucleic acid extraction of DNA a single step should suffice, whereas several steps are required to carry out an on-chip ELISA immunoassay.

FIGS. 2 and 3 thus illustrate embodiments of biological sample processing devices comprising substrate with two area types in the form of areas with hydrophilic and hydrophobic surfaces. The substrate defines an inlet system for receiving the sample and a number of preparation systems for receiving the sample components which have been moved through the oil-filled barrier system.

FIGS. 2 and 3 illustrate examples of highly flexible sample processing devices, where the layout of the first and second area types can be changed by simply changing the structure of the surface modification and the number of fluid reservoirs. Washing steps or other serial operations can be introduced by having more parallel preparation systems between the inlet system and the preparation system where the last step of the particular processing is carried out.

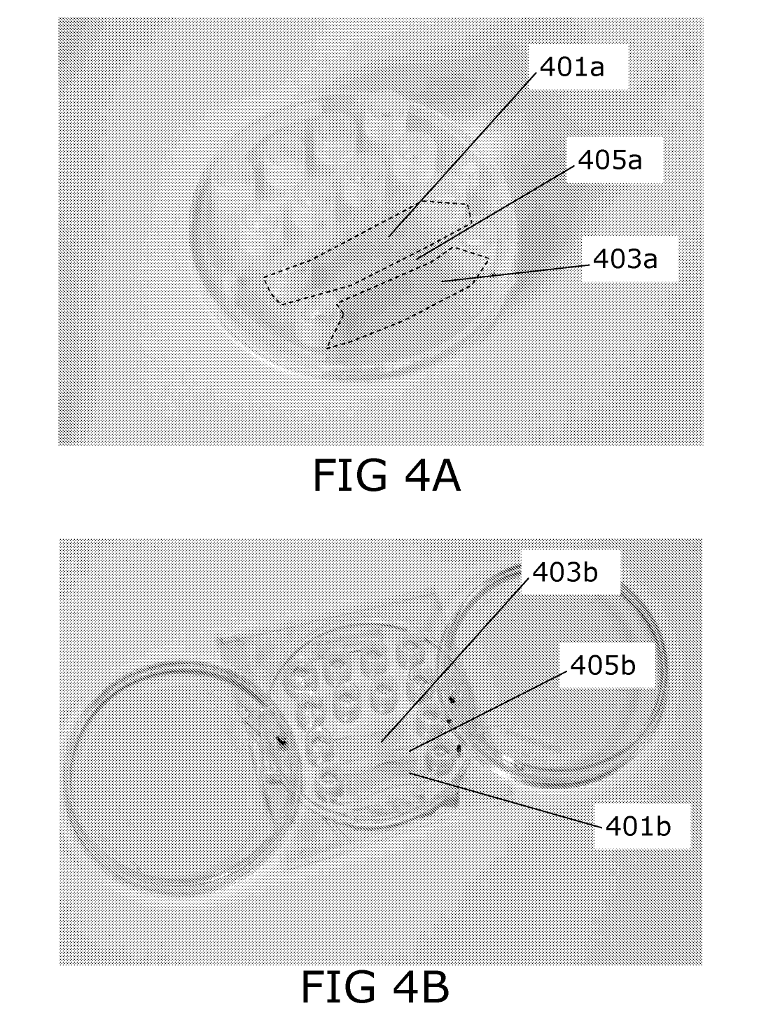

FIG. 4 shows another demonstrator sample processing device.

FIG. 4A shows a sample processing device with a 2 mm thick first substrate which is placed in the bottom in the FIG. 4A. The separation between the first and second substrate is carried out by means of an approximately 250 micrometer thick spacer, which in the present example is an adhesive tape placed in peripheral regions between the first and second substrate. Water with magnetic particles of 1 micrometer in diameter is filled into an inlet system 401a, the water-magnetic particle suspension being brown which in the figure shows as light grey. Water with dissolved copper sulfate is filled into a first preparation system 403a, the water is colored blue by the copper sulfate and this shows up in the figure as a darker phase. An immiscible liquid, which in the present example is hexadecane, is filled into the barrier system 405a.

FIG. 4B shows a sample processing device with a 150 micrometer thick first substrate. A thinner first substrate is likely to entail a stronger magnetic field adjacent to the first surface of the first substrate relative to a magnet on the other side of the first substrate. In the example shown in this figure the separation between first and second substrate is approximately 700 micrometer. As in the example shown in FIG. 4A a spacer is used, the spacer being adhesive tape. The first and second substrate separated by spacers in the form of adhesive tape forms a mechanically stable sample processing device. Water with magnetic particles of 1 micrometer in diameter is filled into an inlet system 401b, the water-magnetic particle suspension being brown which in the figure shows as light grey. Water with dissolved copper sulfate is filled into a first preparation system 403b, the water is colored blue by the copper sulfate and this shows up in the figure as a darker phase. An immiscible liquid, which in the present example is silicone oil, is filled into the barrier system 405b.

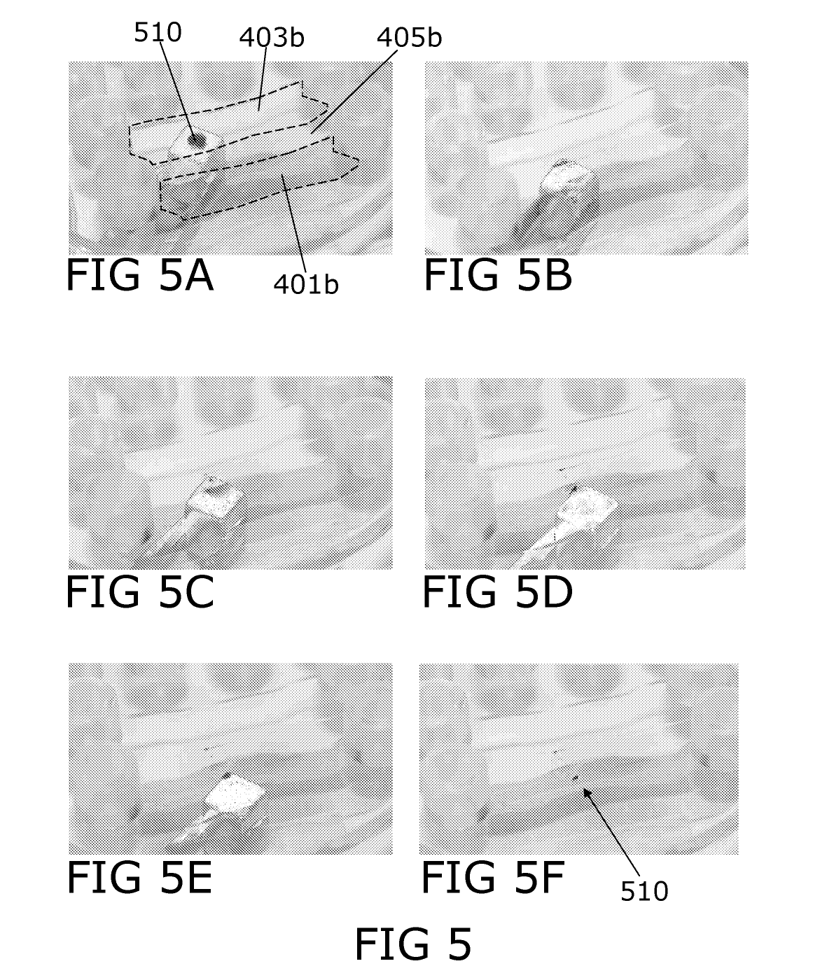

FIG. 5 shows an exemplary movement of magnetic particles, from a first inlet system through the barrier system and into a first preparation system, FIGS. 5A-5F shows snapshots of the process. The sample processing device in FIGS. 5A-5F correspond to the sample processing device in FIG. 4B, with inlet system 401b, first preparation system 403b and barrier system 405b. In FIG. 5A a large number of beads 510 are localized in the inlet system above a permanent magnet, which permanent magnet is held by a tweezer in a position below the first substrate. In FIGS. 5B-5E the beads are moved in consequence of the magnetic field being changed as the magnet held by the tweezer is moved, and in FIG. 5F beads 510 can be seen in the first preparation system, where they show as a spot in the figure. Although in the present example the magnetic field is being changed as the magnet held by the tweezer is moved, in other embodiments, a number of magnetic structures may apply a changing magnetic field so as to change the magnetic field in the systems, and in other embodiments, the magnetic field may be changed in an automated manner, such as an automated moving of one or more magnets.

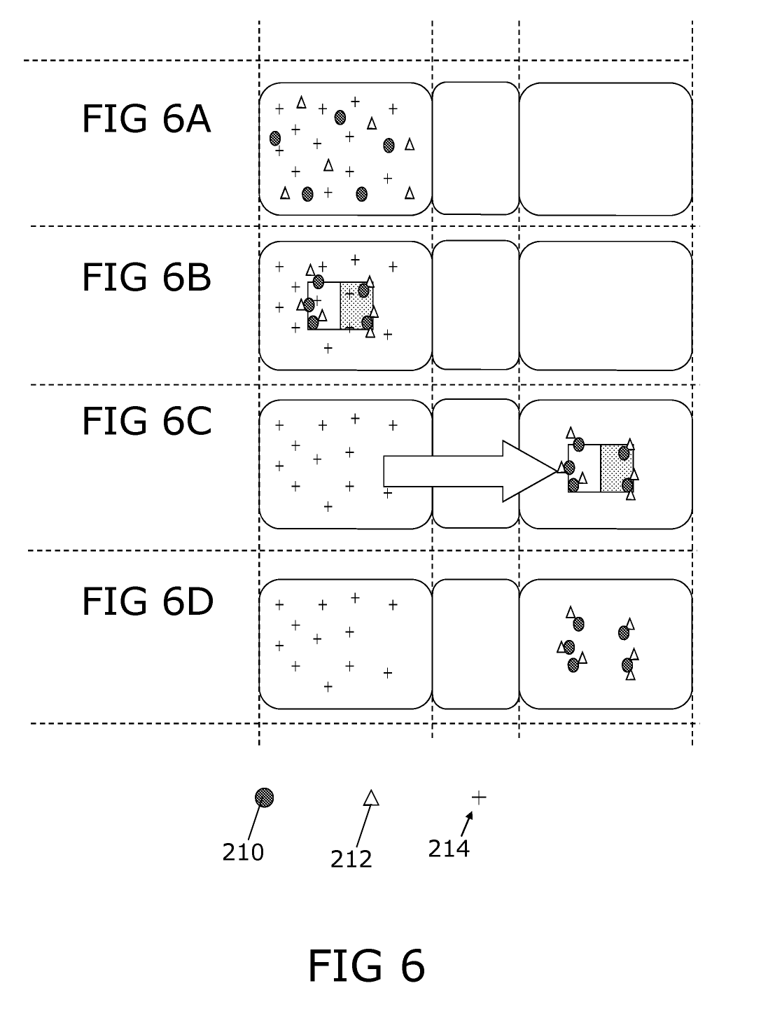

FIG. 6 shows a schematic of magnetic bead translation. FIGS. 6A-6D shows different situations. Each of the FIGS. 6A-6D shows from left to right an inlet system, a barrier system and a first preparation system, each one shown as a rounded rectangle. Although the barrier system is not shown in FIG. 6 to enclose the inlet system and/or the first preparation system, the inlet system and the first preparation system are separated by the barrier system, so that sample transport between the inlet system and the first preparation system does not occur by itself, such as by diffusion, on a time-scale corresponding to the time for processing a sample. The three sides of the inlet system and the first preparation system system which are not bordering the barrier system may in an exemplary embodiment border a wall structure, such as a surface being orthogonal to the plane of the first surface of the first substrate. Likewise with two sides of the barrier system which are not bordering respectively the inlet system and the first preparation system. The symbols shown below FIG. 6D are a magnetic bead 210, a specific sample 212 and a non-specific sample 214. In FIG. 6A is shown an initial situation. The leftmost rounded rectangle illustrates an inlet system, which inlet system comprises a sample liquid with magnetic beads, non-specific samples and specific samples. FIG. 6B shows a situation where magnetic capture is occurring, as illustrated with a magnet. Typically, the magnet is placed on the other side of either the first or second substrate, with respect to the sides of the first or second substrate which is facing the other substrate. The magnetic field resulting from the magnet serves to localize the magnetic beads close to the magnet. The figure also shows that the specific samples are bound by the magnetic beads. In one example, the magnetic beads may be coated with a material, such as a molecular layer, which is capable of selectively binding the specific samples. FIG. 6C shows magnetic bead translation, where magnetic beads are moved from the inlet system, through the barrier system as indicated by the arrow, and into the preparation system. FIG. 6D shows release of the magnetic beads. This release may, for example, be induced by removing the magnetic field, such as by removing the magnet.

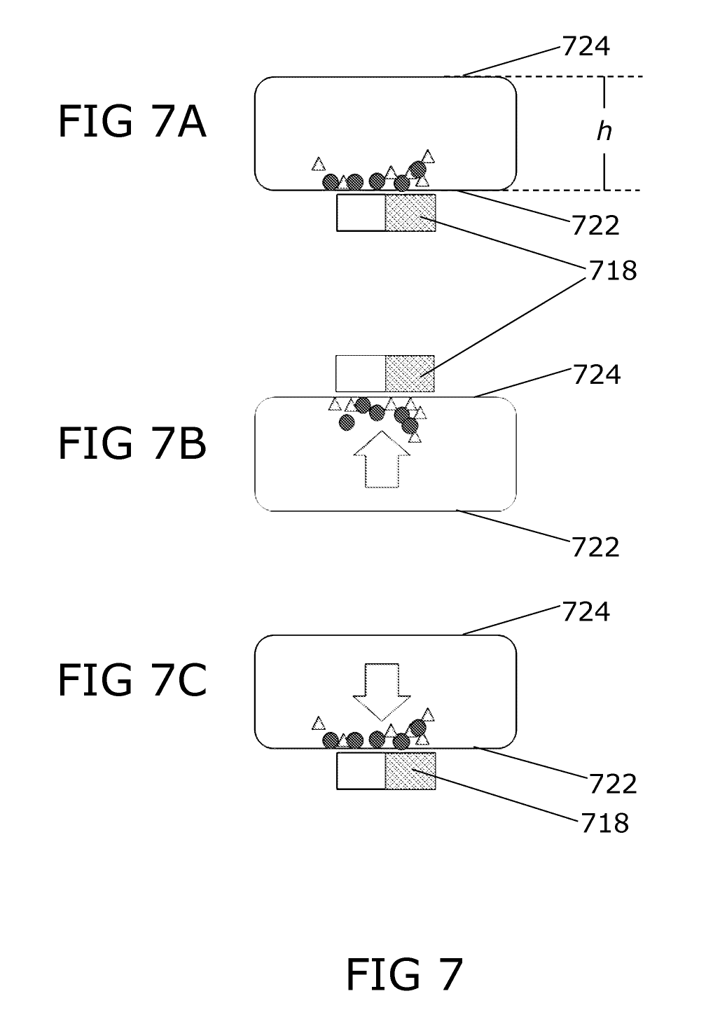

FIG. 7 shows a side view schematic of magnetic bead capture. In FIG. 7A, the magnetic beads (depicted as filled circles, according to the schematics of FIG. 6) are assembled at the bottom of the system, which system may be any one of an inlet system, a barrier system or a first preparation system. Specific samples (depicted as triangles, according to the schematics of FIG. 6) are bound to the magnetic beads. The bottom region of the system may be adjacent to the first surface 722 of the first substrate, whereas the top region may correspond to the second surface 724 of the second substrate. A magnet 718 on the other side of the first substrate with respect to the first surface 722 which, faces the second substrate, is attracting the magnetic beads to the bottom region of the system. The distance between the first surface 722 and the second surface 724 is denoted h. FIG. 7B shows a magnet 718 placed above the system, and hence moving the magnetic beads to a top region. FIG. 7C shows moving of the magnetic beads back to the bottom region by placing the magnet 718 below the system.

FIG. 8 shows a schematic with various lengths indicated.

FIG. 8A shows in an exemplary embodiment a straight inlet system. The volume of a typical sample to be analyzed may be estimated as follows. If the inlet system width is w=5 mm and the distance between the first and second substrate, corresponding to height h, is h=1 mm, the volume of a square as seen from the top is w×w×h=25 microlitre. Choosing a magnet size slightly smaller than the inlet system width, w, this is the volume from which we can expect to capture the magnetic beads by use of the magnet.

FIG. 8B shows another inlet system. By expanding the length of the inlet system as illustrated in FIG. 8B and sweeping the magnet along the length of the inlet system, this volume can reasonably be expanded to a length of about L=15 mm such that the total sample volume can be estimated to be about L×w×h=75 microlitre, which is slightly larger than the typical sample volumes used in microtitre plates. Thus, analyses that are currently carried out in microtitre plates should be feasible with sample processing device—albeit with the possible advantage of simplified sample handling, reduced analysis time (as diffusion is not necessarily relied upon) and potentially smaller sample loss during washing.

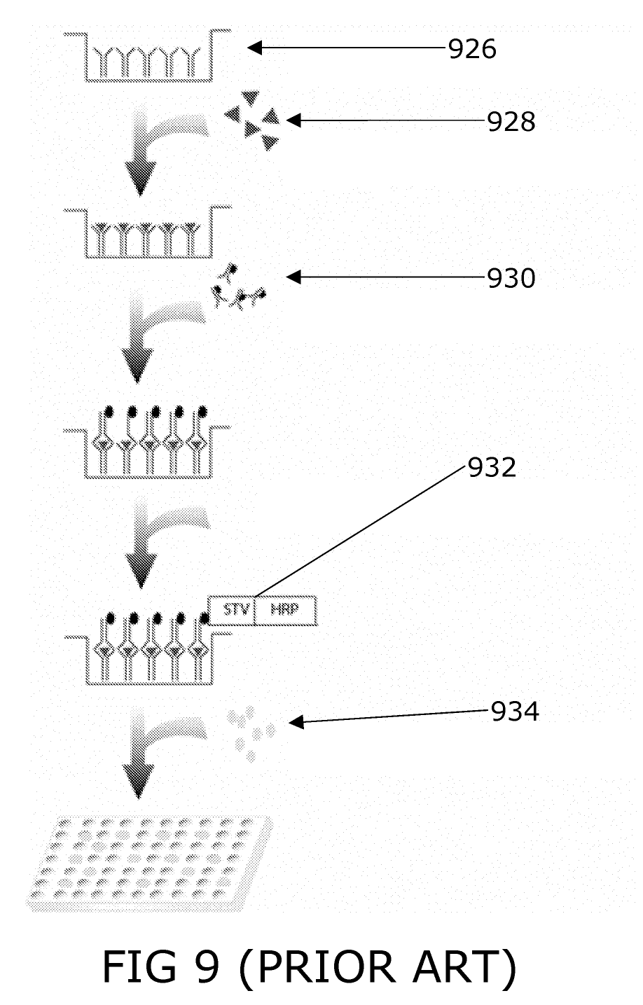

FIG. 9 shows a schematic illustration of a standard ELISA procedure as is generally known in the art. In the shown ELISA procedure, the starting point is a plate coated with capture antibody 926. Then sequentially samples 928, biotin-labeled detection antibody 930, streptavidin HRP (Horse Radish Peroxidase) 932 and substrate 934 is added. The steps of the procedure which may be carried out in a microtitre plate with wells are summarized in table I.

TABLE I

| Step # | | Task | Time | |
|---|---|---|---|---|
| Plate | 1 | Incubate: | 12 | hrs |
| coating | | 100 μL capture antibody (5 μg/mL) | | |
| | | overnight at 4° C. | | |
| | 2-4 | Wash: | 5 | min |
| | | 3× with PBS-T | | |
| | 5 | Block: | 1 | hr |
| | | 300 μL blocking buffer (PBS with | | |
| | | 1 wt % BSA, 5 wt % sucrose, 0.05 | | |
| | | wt % sodium azide) for 1 hour | | |
| sample | 6 | Incubate: | 15-120 | min |
| incuba- | | sample/antigen for 15 minutes | | |
| tion | | to 2 hours | | |
| | 7-9 | Wash: | 5 | min |
| | | 3× with PBS-T | | |
| +detection | 10 | Incubate: | 60 | min |
| Ab | | 100 μL biotin-conjugated | | |
| | | detection antibody (200 ng/mL) | | |
| | | for 1 hour | | |
| | 11-13 | Wash: | 5 | min |
| | | 3× with PBS-T | | |
| +enzyme | 14 | Incubate: | 30 | min |
| (HRP) | | 100 μL of SA-HRP (5 μg/mL) | | |
| | | for 30 minutes | | |
| | 15-17 | Wash: | 5 | min |
| | | 3× with PBS-T | | |
| +substrate, | 18 | Incubate: | 20 | min |
| read-out | | 100 μL TMB substrate for | | |
| | | exactly 20 minutes. | | |
| | 19 | Read-out: | 5 | min |
| | | Either | | |
| | | (1) measure the absorption at | | |
| | | 605 nm or (2) add 100 μL | | |
| | | H₂SO₄ (1M) and measure the | | |
| | | absorption at 450 nm | | |

The materials used include:

### PBS: Phosphate-Buffered Saline

PBS-T: PBS with added 0.05 wt % Tween-20

### BSA: Bovine Serum Albumin

### SA-HRP: Streptavidin-Horse Radish Peroxidase

### TMB: Tetra-Methyl-Benzidine

Every step in the sequence given in Table I (except step 19) corresponds to an exchange of the fluid in the well. In many settings, one will use a pre-coated plate such that steps 1-5 can be omitted at the site of analysis. The main factor limiting the time of steps 6-19 is the incubation, where long times are needed to ensure diffusion of the samples to the wall of the plate. The total time for steps 6-19 may be estimated to be 145-250 minutes.

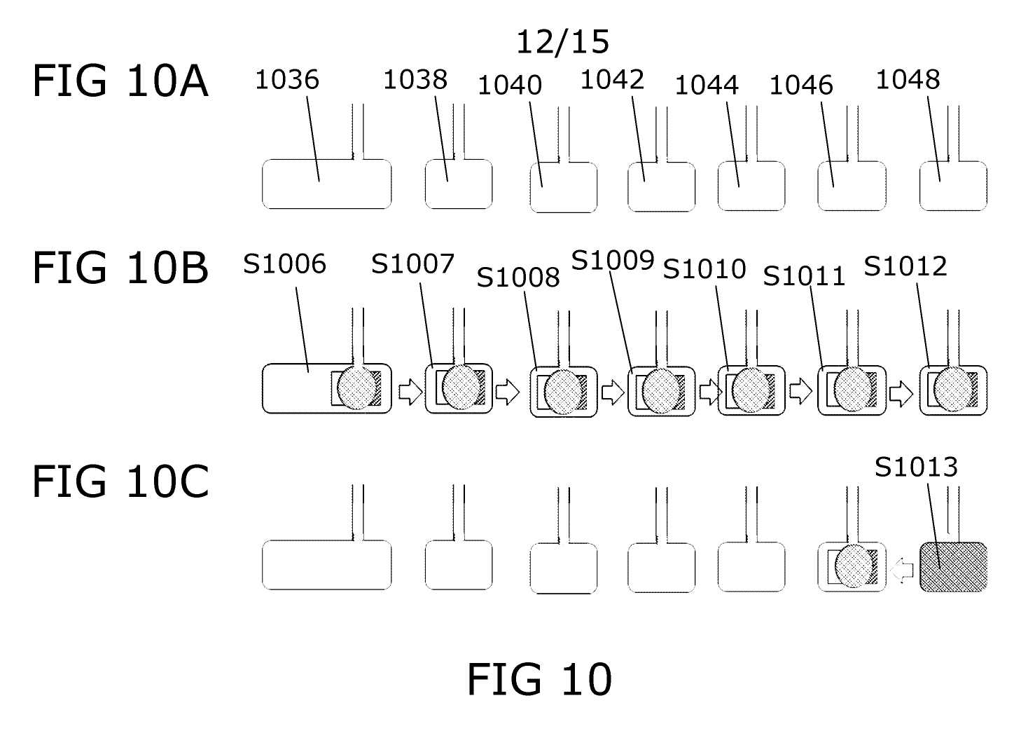

FIG. 10 shows a schematic illustration of a sample processing device based implementation of an ELISA procedure for single sample analysis. FIG. 10A shows a layout of an initial system with an inlet system 1036 with magnetic beads coated with capture antibody. A first preparation system 1038 contains buffer (PBS-T) for washing, further preparation systems 1040-1048 contains respectively detection antibody, buffer (PBS-T) for washing, streptavidin-Horse Radish Peroxidase, buffer (PBS-T) for washing and TMB substrate. FIG. 10B shows a method of performing ELISA single sample analysis with the sample processing device. Steps S1006-S10012 corresponds to steps 6-12 in Table II. FIG. 10C illustrates read out S1013 corresponding to step 13 in Table II. Several aspects of the embodiment depicted in FIG. 10 are addressed in the following:

### Sample Processing Device Dimensions

The sketch in FIG. 10 shows a single horizontal chain of systems, such as inlet system and preparation systems. If the typical width of an inlet system and/or a preparation system, respectively, and spacing between the systems is about 5 mm, the total horizontal dimension of the sample processing device is about 70 mm (and can fit onto a glass slide dimension). These dimensions, however, can be significantly reduced, if needed. Several of these chains can be repeated in the vertical direction in the figures.

### Reagent Filling

The reagents can be introduced by capillary forces through separate inlets or the dry constituents of the reagents can be freeze-dried on the sample processing device, such as on the first and/or second surface and mixed with water introduced through a single inlet connected to all the preparation systems. First, the sample processing device will be filled with the reagents.

### Sample Filling

Next the sample liquid is introduced and it fills the inlet system by capillary forces. The sample liquid can either be premixed with beads coated with the capture antibody or these beads can be stored in dry form in the inlet system on the sample processing device.

### Immiscible Phase Filling

Finally, if needed, the liquid phase immiscible with water is introduced. This phase will wet and fill the remaining areas on the sample processing device. Proper venting holes may be included in the design to ensure pressure equilibration (otherwise the phase may be prevented from wetting parts of the surface, such as cover parts of the surface). If no liquid phase immiscible with water is needed and the barrier system is simply filled with air instead, this potential issue is of minor concern.

### Assay Procedure (Steps 6-12)

The assay procedure is schematically listed in Table II. At each step where beads are moved, they will be actively mixed with the liquid in the compartment (as indicated).

TABLE II

| Step # | | Task | Time | |
|---|---|---|---|---|
| Bead | 1 | Incubate: | 12 | hrs |
| coating | | 100 μL capture antibody (5 μg/mL) | | |
| with Ab's | | overnight at 4° C. | | |
| (details to | 2-4 | Wash: | 5 | min |
| be deter- | | 3× with PBS-T | | |
| mined) | 5 | Block: | 1 | hr |
| | | 300 μL blocking buffer (PBS with | | |
| | | 1 wt % BSA, 5 wt % sucrose, 0.05 | | |
| | | wt % sodium azide) for 1 hour | | |
| sample | 6 | Incubate with active magnetic mixing: | 5 | min |
| incuba- | | sample/antigen | | |
| tion | 7 | Wash with active magnetic mixing: | 5 | min |
| | | 1× with PBS-T | | |
| +detection | 8 | Incubate with active magnetic mixing: | 5 | min |
| biotin-Ab | | 100 μL biotin-conjugated detection | | |
| | | antibody (200 ng/mL) | | |
| | 9 | Wash: | 5 | min |
| | | 1× with PBS-T | | |
| +enzyme | 10 | Incubate with active magnetic mixing: | 5 | min |
| (SA-HRP) | | 100 μL of SA-HRP (5 μg/mL) | | |
| | 11 | Wash with active magnetic mixing: | 5 | min |
| | | 1× with PBS-T | | |
| +substrate, | 12 | Incubate with active magnetic mixing: | 5 | min |
| read-out | | 100 μL TMB substrate for exactly 5 min. | | |
| | 13 | Read-out: | 5 | min |
| | | measure the absorption at 605 nm | | |

The total time for steps 6-13 may be estimated to be 40 minutes, such as the total time for steps 6-13 being estimated to be less than 40 minutes.

It is noted, that possible advantages of this embodiment of the sample processing device includes the following:
As the magnetic beads can be actively mixed with the fluid, such as by stirring, such as by using a magnetic sample manipulation component for stirring, the procedure does no longer rely on diffusion of the samples over long distances. This significantly reduces the assay time.
Due to the translation of the beads from one compartment to another through a phase (oil or air) immiscible with water and the active manipulation of beads during washing, it is anticipated that less washing is needed compared to the standard ELISA protocol. Therefore, only a single washing step has been included in the schematic. Potentially, the washing steps (steps 7, 9 and 11) can be removed due to the immiscible phase filter and this would greatly simplify the process and the area requirements (only three preparation systems would be needed).
The total surface area of the beads is likely to be larger than the surface area of the ELISA well. This may result in a higher sensitivity.

### Read-Out (Step 13)

The read-out can be (e.g.) based on various methods, including methods based on electrochemistry, optics or radioactivity. FIG. 10 assumes optical read-out. After generation of the coloured product, the beads are captured and removed from the preparation system. This ensures that no beads or magnet structures are in the optical path. The optical path may be along a line traversing the second substrate, the distance between the first and second substrate and the first substrate, the line being substantially orthogonal to the plane of the first and second surface. The reaction products are then quantified by absorbance measurements through the system comprising the reaction products. A comment on optical transparency of COC/Topas: above about 400-500 nm the transmittance through a 2 mm thick piece of COC/Topas is about 90%, so optical detection through a sample processing device having first and second substrates comprising COC/Topas seems feasible.

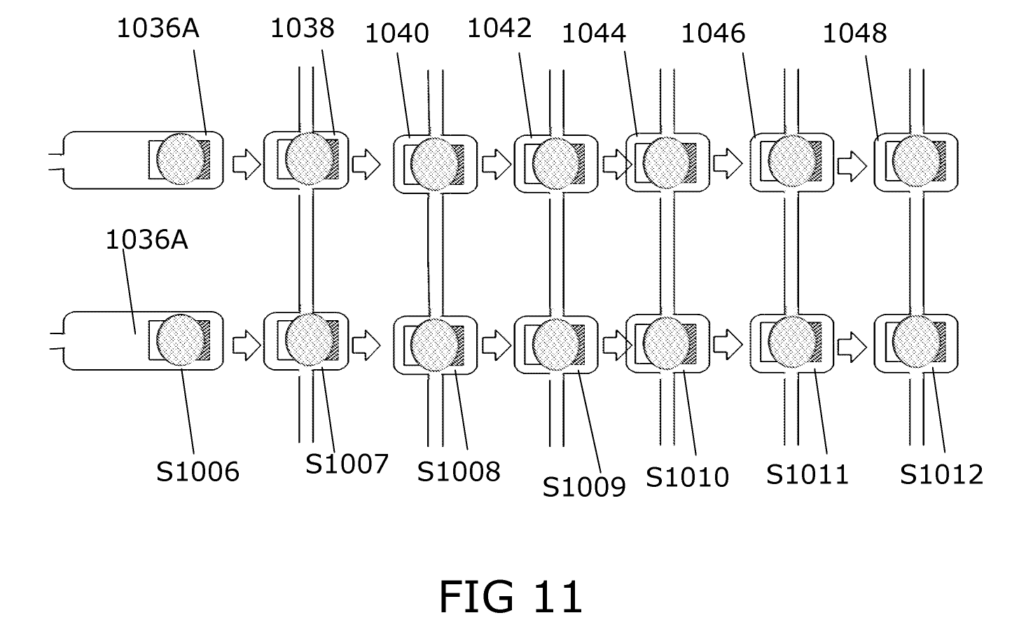

FIG. 11 shows a schematic illustration of a sample processing device based implementation of a standard ELISA procedure for multiple sample liquids, single sample analysis. Specifically, the figure shows a possible sample processing device layout for the simultaneous analysis of a single sample in multiple sample liquids. In the figure, two samples can be seen in two separate inlet systems 1036A and 1036B. The preparation systems 1038-1048 and steps S1006-S1012 are similar to those described in connection with FIG. 10. Notice that for clarity, reference signs 1038-1048 are only shown for the upper row of system and reference signs S1006-S10012 are only shown for the lower row of systems which in this context represents method steps. However, in FIG. 11 two samples are treated in parallel. Several aspects of this embodiment are addressed in the following:

### Expansion to Multiple Samples or Reagents

The sample processing device for single sample analysis presented in FIG. 10 can be expanded to analyze several sample liquids (including reference sample liquids) and/or several samples.

### Several Samples

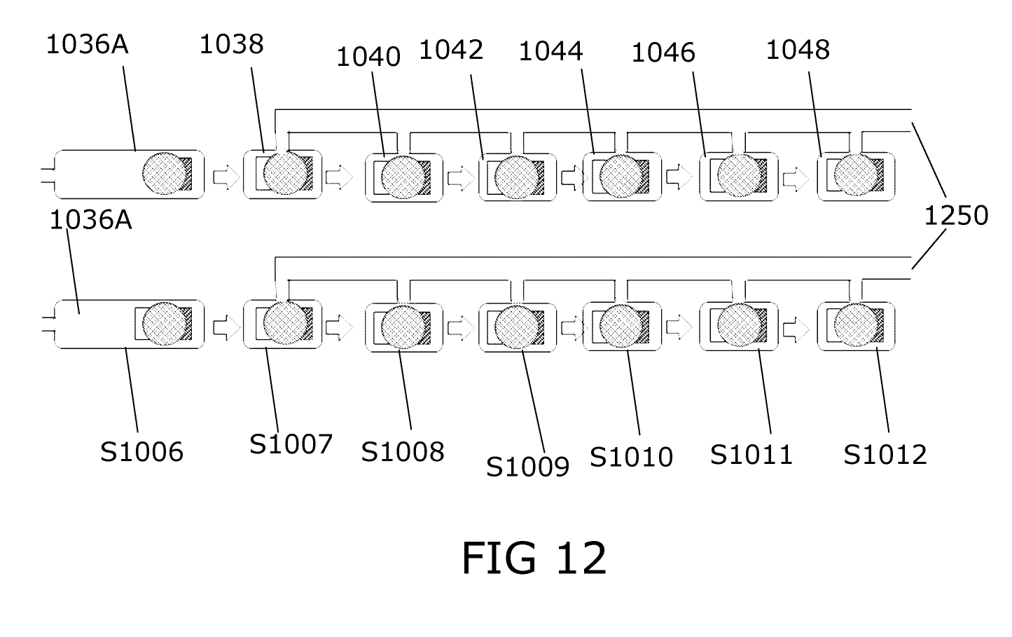

One way to analyze several sample liquids for the same sample is to have inlet systems that are separated from each other and fluidically connected to different inlets on the sample processing device. The magnetic beads can then either be premixed with each sample in each sample liquid or they can be stored in dry form in each inlet system. The preparation systems can share the same inlet as illustrated in FIG. 11 (for example, if the reagents are loaded in liquid form). It is noted that care must be taken to ensure that there is no cross-talk between adjacent systems in the vertical direction. One method to achieve this may be to ensure that the channel areas connecting the preparation systems are so thin, such as narrow, and so long, that diffusion between the preparation systems, such as diffusion of reagents and/or sample is substantially negligible on a time scale of a processing on the processing device during use. Alternatively, each preparation system can have the reagents stored in dry form and an aqueous phase, such as water, is introduced from a common inlet (shown in FIG. 12).

FIG. 12 shows a schematic illustration of a sample processing device based implementation of a standard ELISA procedure for multiple samples, multiple sample analysis. The preparation systems 1038-1048 and steps S1006-S1012 are similar to those described in connection with FIG. 10. Notice that for clarity, reference signs 1038-1048 are only shown for the upper row of system and reference signs S1006-S10012 are only shown for the lower row of systems which in this context represent method steps. Specifically, the figure shows a possible sample processing device layout for the simultaneous analysis of several samples in multiple sample liquids. In this case, the beads will have to be stored in dry form in part of the inlet system and the reagents (different detector Ab's) will have to be stored in dry form in the relevant preparations systems as illustrated in FIG. 12. The aqueous phase for the reagents can then be introduced through a common inlet 1250 connected to the preparation systems.

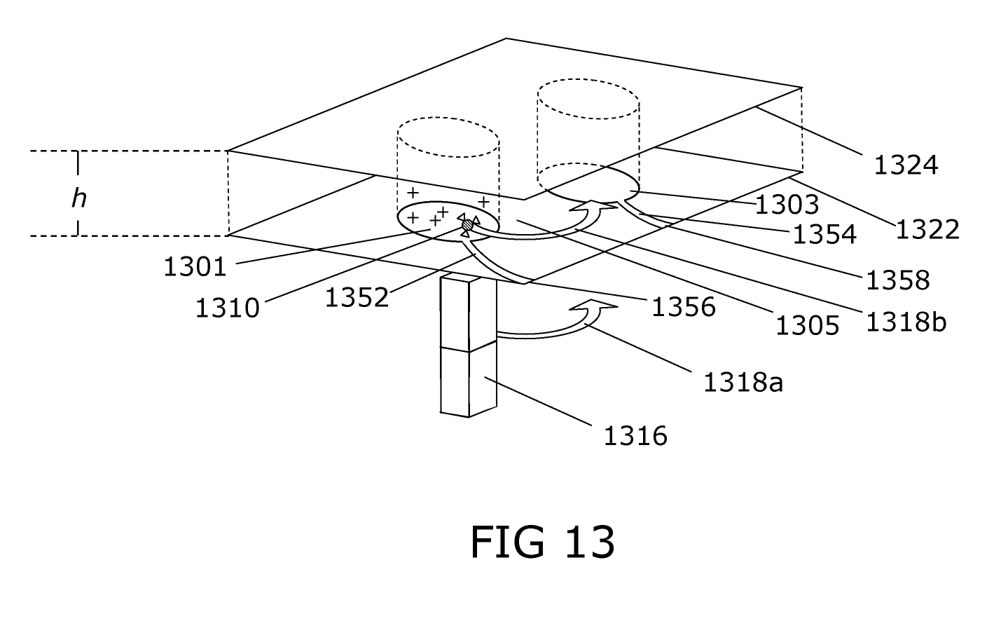

FIG. 13 shows a perspective schematic drawing of an embodiment of a sample processing device according to an embodiment of the invention, the sample processing device comprising a first substrate, the first substrate having a first surface 1322 comprising two area types, a first area type where the first surface is hydrophilic indicated by regions corresponding to an inlet system 1301 and a first preparation system 1303 and a second area type where the first surface is hydrophobic which in the present embodiment is the remaining part of the first surface 1322, and a second substrate, the second substrate having a second surface 1324 positioned substantially parallel with the first surface at a distance h from the first surface 1322 of the first substrate; the first substrate and the second substrate defines: an inlet system 1301 provided in an area of the first type; a first preparation system 1303 provided in an area of the first type; and a barrier system 1305 provided in an area of the second type; wherein the inlet system 1301 and the first preparation system 1303 are separated by the barrier system, and wherein the inlet system 1301 is adapted to receive a sample liquid comprising the sample and the first preparation system 1303 is adapted to receive a receiving liquid. During use, a sample liquid comprising a sample may be provided in the inlet system 1301 corresponding to a volume between the first surface and the second surface whose projection onto the first surface corresponds to an area of the first area type; a receiving liquid may be provided in the first preparation system 1303 corresponding to a volume between the first surface and the second surface whose projection onto the first surface corresponds to an area of the first area type; and moving sample through the barrier system to the first preparation system to generate a processed sample. The sample liquid may be provided in the inlet system 1301 via a channel area 1352 of the first area type, wherein the channel area is an area of the first type arranged so that an aqueous phase may be driven by capillary forces from an inlet, such as an inlet 1356 between the first and second substrate on the lateral edge of the first and second substrate. In the shown embodiment, the channel area comprises a relatively thin area with respect to the dimensions of the inlet system in the plane of the first substrate. Similarly, the receiving liquid may be provided in the first preparation system 1303 via a channel area 1354 of the first area type, wherein the channel area is an area of the first type arranged so that an aqueous phase may be driven by capillary forces from an inlet, such as an inlet 1356 between the first and second substrate. The inlets to each of the channel areas 1352, 1354 may also be embodied as through-going holes in the first or second substrate. In an alternative embodiment, inlets may be fluidically connected directly to the inlet system and/or the first preparation system. In the particular embodiment depicted in FIG. 13, the sample is depicted with triangles and attached to magnetic particles 1310 (same symbols as in FIG. 2), which can be moved by changing a magnetic field, such as moving a permanent magnet 1316 along a trajectory 1318a parallel with the plane of the first surface, whereby the magnetic particles 1310 are moved along a corresponding trajectory 1318b. Notice that the depicted trajectory is comprised within a plane which is parallel to the first and second surface. The trajectory is non-linear, but could have been linear so that the magnetic bead would have followed a rectiliniear path from the inlet system, through the barrier system to the first preparation system. The linear motion is very simple and thus easy to carry out manually or to automate. Furthermore, more preparation systems could be added to the device, and the motion could intersect these preparation systems as well, so as to realize a serial processing of the sample in a simple manner.

To sum up, a sample processing device is disclosed, which sample processing device comprises a first substrate and a second substrate, where the first substrate has a first surface comprising two area types, a first area type where the first surface is hydrophilic and a second area type where the first surface is hydrophobic. The first substrate defines an inlet system and a preparation system in areas of the first type which two areas are separated by a barrier system in an area of the second type. The inlet system is adapted to receive a sample liquid comprising the sample and the first preparation system is adapted to receive a receiving liquid. In a particular embodiment, a magnetic sample transport component, such as a permanent magnet or an electromagnet, is arranged to move magnetic beads in between the first and second substrates.

In one exemplary embodiment, there is provided:

E1. A sample processing device comprising:
a first substrate, the first substrate having a first surface, and;
a second substrate, the second substrate having a second surface positioned substantially parallel with the first surface at a distance from the first surface of the first substrate;
   the first surface of the first substrate and the second surface of the second substrate comprising two area types, a first area type being hydrophilic and a second area type being hydrophobic; the first substrate and the second substrate defines:
an inlet system provided in an area of the first type;
a first preparation system provided in an area of the first type; and
a barrier system provided in an area of the second type;
   wherein the inlet system and the first preparation system are separated by the barrier system, and wherein the inlet system is adapted to receive a sample liquid comprising the sample and the first preparation system is adapted to receive a receiving liquid.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A sample processing device comprising:
a first substrate, the first substrate having a first surface comprising at least two area types, a first area type with a first contact angle with water and a second area type with a second contact angle with water, the first contact angle being smaller than the second contact angle, and;
a second substrate, the second substrate having a second surface positioned substantially parallel with the first surface at a distance from the first surface of the first substrate;
the first surface of the first substrate, or the first surface of the first substrate and the second surface of the second substrate, defines:
an inlet system provided in an area of the first type;
a first preparation system provided in an area of the first type; and
a barrier system provided in an area of the second type;
wherein the inlet system and the first preparation system are separated by the barrier system, and wherein the inlet system is adapted to receive a sample liquid comprising the sample and the first preparation system is adapted to receive a receiving liquid.

2-19. (canceled)

20. The sample processing device according to claim 1, wherein the first area type is hydrophilic and the second area type is hydrophobic.

21. The sample processing device according to claim 1, wherein the difference in contact angle between the first contact angle of the first area type and the second contact angle of the second area type enables filling the inlet system and first preparation system with an aqueous solution and subsequent filling the barrier system with a liquid immiscible with the aqueous solution whereby the position of the aqueous phase and the liquid immiscible with the aqueous phase are defined by the positions of the first and second area types.

22. The sample processing device according to claim 1, wherein the barrier system is adapted to receive a liquid immiscible with an aqueous phase.

23. The sample processing device according to claim 22, wherein the water immiscible liquid is selected from the group consisting of oil, wax, ionic liquids, alcohols, amines, carboxylic acids, esters, amides, and ketones, wherein the alcohols, amines, carboxylic acids, esters, amides, or ketones have a chemical structure containing a plurality of carbon atoms.

24. The sample processing device according to claim 1 comprising two or more preparation systems, each preparation system being separated by the barrier system.

25. The sample processing device according to claim 1, wherein the first preparation system is pre-filled with a reagent.

26. The sample processing device according to claim 25, wherein the pre-filled reagent is selected from the group consisting of a dried reagent, a freeze dried reagent, a reagent contained in a gel and a reagent contained in a liquid.

27. The sample processing device according to claim 1, wherein a sample may be moved from the inlet system to the first preparation system through non-solid matter, whereby the sample is moved along a trajectory substantially confined to a plane.

28. The sample processing device according to claim 1, further comprising a magnetic sample transport component arranged to move magnetic particles between the inlet system and the first preparation system and/or between two preparation systems.

29. The sample processing device according to claim 1, further comprising a magnetic sample manipulation component arranged to move magnetic particles from a starting point to an end point along a path, the path being within the inlet system or within the barrier system or within the first preparation system, wherein the length of the path is substantially larger than a distance from the starting point to the end point.

30. The sample processing device according to claim 1, further comprising a fluid reservoir connected to any one of: the inlet system, the first preparation system, the barrier system, and wherein the inlet system, the preparation system and/or the barrier system is dimensioned so that fluid is pulled from the fluid reservoir to the inlet system, the preparation system and/or the barrier system by capillary forces.

31. The sample processing device according to claim 1, wherein the barrier system is fluidically connected to a least one venting means, the venting means allowing passage of a fluid from within any one of
the inlet system,
the barrier system,
the first preparation system,
and/or through the first substrate and/or through the second substrate and/or between the first substrate and the second substrate.

32. A method of processing a sample on a sample processing device, the sample processing device comprising:
a first substrate, the first substrate having a first surface comprising two area types, a first area type with a first contact angle with water and a second area type with a second contact angle with water, the first contact angle being smaller than the second contact angle;
the first substrate defines:
an inlet system provided in an area of the first type;
a first preparation system provided in an area of the first type; and
a barrier system provided in an area of the second type;
wherein the inlet system and the first preparation system are separated by the barrier system, and wherein the inlet system is adapted to receive a sample liquid comprising the sample and the first preparation system is adapted to receive a receiving liquid, the method comprising:
providing the sample liquid comprising a sample in the inlet system;
providing the receiving liquid in the first preparation system; and
moving the sample through the barrier system to the first preparation system to generate a processed sample.

33. A method according to claim 32, wherein the first surface is hydrophilic in the first area type and the first surface is hydrophobic in the second area type.

34. The method according to claim 32, wherein the inlet system comprises magnetic beads with associated molecules, and wherein the movement of the sample through the barrier system is done by moving the magnetic sample transport component to move the sample through the barrier system.

35. The method according to claim 32, wherein prior to moving the sample through the barrier system, a water immiscible liquid is provided in the barrier system.

36. The method according to claim 32, further comprising moving the processed sample through the barrier system from the first preparation system to a second preparation system to generate a further processed sample.

37. The method according to claim 36, wherein moving the processed sample is done along a trajectory, the trajectory being substantially confined to a plane.

38. The method according to claim 32, wherein the sample contains at least one component selected from the group consisting of: cells, intact cells, virus, nucleic acids, peptides, proteins, and small organic molecules, or small organic molecules that are toxic to the environment, animals, plants, and/or humans.
